(19) United States
(12) Patent Application Publication
(10) Pub. No.: US 2023/0323422 A1
(43) Pub. Date: Oct. 12, 2023

(54) METHOD OF MAKING PROTEIN

(71) Applicant: Bristol-Myers Squibb Company, Princeton, NJ (US)

(72) Inventors: Amanda Morgan LEWIS, Waltham, MA (US); Alison Gene-Wei LEE, Austin, TX (US); Nancy Ann LAMON, Bedford, NH (US); Terrance Wayne CARONE, II, Liverpool, NY (US); Ronald Charles BATES, Irvine, CA (US); Richard Thomas LUDWIG, Hopewell, NJ (US)

(73) Assignee: Bristol-Myers Squibb Company, Princeton, NJ (US)

(21) Appl. No.: 18/041,665

(22) PCT Filed: Aug. 13, 2021
(86) PCT No.: PCT/US2021/045959

§ 371 Date: Feb. 14, 2023

Related U.S. Application Data
(60) Provisional application No. 63/199,547, filed on Jan. 7, 2021.
(60) Provisional application No. 63/066,127, filed on Aug. 14, 2020.

(51) Int. Cl. C12P 21/02; C12P 21/00; G01N 33/68; C07K 14/705

(52) U.S. Cl. CPC C12P 21/02; C12P 21/005; G01N 33/68; C07K 14/70521; C07K 2319/30; G01N 2400/38

(57) ABSTRACT

This disclosure provides a novel method of controlling the glycosylation profile of a protein during production. The disclosure also provides a novel method of improving protein yield while controlling the glycosylation profile of a protein.

### CROSS-REFERENCE TO RELATED APPLICATIONS

This application claims priority benefit of U.S. Provisional Application Nos. 63/066,127, filed Aug. 14, 2020, and 63/199,547, filed Jan. 7, 2021, each of which is herein incorporated by reference in its entirety.

### REFERENCE TO SEQUENCE LISTING SUBMITTED ELECTRONICALLY VIA EFS-WEB

The content of the electronically submitted sequence listing in ASCII text file (Name 3338_205PC02_Seglisting_ST25.txt; Size: 25,078 bytes; and Date of Creation: Aug. 10, 2021), filed with the application, is incorporated herein by reference in its entirety BACKGROUND OF THE DISCLOSURE

The market for protein therapeutics has grown significantly and the pace of development continues to increase. It is a challenge, however, for the industry to maintain the desired quality attributes, e.g., glycosylation, while maintaining the quantity, reducing the cost of production, and providing production flexibility. Efficient manufacturing scale production of protein therapeutics is required to continue to meet the needs of the market.

Glycosylation is one of the most abundant of all protein post-translational modifications (PTMs). It results from the addition of sugar residues to protein sidechains to form a glycoprotein. Mammalian glycoprotein oligosaccharides are commonly built from a limited number of monosaccharides but their structural diversity is vast, mainly because they often form complex branching patterns.

Glycosylation plays an important role in many specific biological functions, including immune defense, fertilization, viral replication, parasitic infection, cell growth, inflammation, and cell-cell adhesion. For pharmaceutical glycoproteins, glycosylation affects stability of protein conformation, clearance rate, protection from proteolysis, and improves protein solubility. Since different glycoforms have the potential to have different biological properties, the ability to monitor and control glycosylation during production is critical to the quality of a biopharmaceutical molecule.

However, glycosylation naturally occurs with a certain degree of heterogeneity and can be affected by many different factors, such as the expression system, process conditions, media composition, feed protocols, purification process, or any combination thereof Consequently, there are needs to improve the protein manufacturing process including culturing and/or purification while maintaining the glycosylation pattern of the protein consistent.

### SUMMARY OF THE DISCLOSURE

The present disclosure is related to a method of improving the yield of a protein and/or controlling a glycosylation of the protein during a protein production phase, comprising culturing cells that are capable of expressing the protein in a bioreactor for a protein induction phase under suitable conditions, wherein the suitable conditions comprise a pH set point between about 7.1 and about 7.2. In some aspects, the pH set point is about 7.15. In some aspects, the suitable conditions further comprise an initial temperature set point between about 35° C. and about 37° C., a second temperature set point between about 32° C. and about 34° C., and a third temperature set point between about 30° C. to about 32° C. In some aspects, the suitable conditions further comprise an initial viable cell density (VCD) set point between about 0.5×10⁶ cells/mL and about 1×10⁶ cells/mL. In some aspects, the suitable conditions comprise: (a) an initial temperature set point of about 36° C., a second temperature set point of about 33° C., and a third temperature set point of about 31° C.; (b) a pH set point of about 7.15; and (c) an initial viable cell density (VCD) set point of about 0.70×10⁶ cells/mL.

The present disclosure is related to a method of controlling cell growth rate, cell viability, viable cell density and/or titer of cells for producing a protein comprising culturing the cells in a bioreactor for a protein induction phase under a pH set point of about 7.15. In some aspects, the suitable conditions further comprise: (i) an initial temperature set point of about 36.0° C. and a second temperature set point lower than about 36° C.; (ii) an initial temperature set point lower than about 36.5° C. and a final temperature set point of about 31° C.; or (iii) an initial temperature set point lower than about 36.5° C., a second temperature set point of about 33° C., and a final temperature set point lower than about 33° C. In some aspects, the suitable conditions further comprise culturing the cell in an initial temperature set point of about 36° C., a second temperature set point of about 33° C., and a final temperature set point of about 31° C. In some aspects, the suitable conditions further comprise an initial viable cell density (VCD) set point of about 0.70×10⁶ cells/mL.

The present disclosure is also related to a method of improving the yield of belatacept and/or controlling the glycosylation of belatacept during a protein production phase, comprising culturing cells that are capable of expression belatacept in a bioreactor under suitable conditions, wherein the suitable conditions comprise: (a) an initial temperature set point of about 36° C., a second temperature set point of about 33° C., and a third temperature set point of about 31° C.; (b) a pH set point of about 7.15; and (c) an initial viable cell density (VCD) set point of about 0.70×10⁶ cells/mL.

In some aspects, the suitable conditions further comprise a first feed time at about 80 hours. In some aspects, the third or final temperature set point occurs between about 204 and about 276 hours. In some aspects, third or final temperature set point occurs at about 204 hours, about 216 hours, about 228 hours, about 240 hours, about 252 hours, about 264 hours, or about 276 hours after the initial temperature set point. In some aspects, the third final temperature set point is about 31° C. and occurs after about 240 hours. In some aspects, the second temperature set point occurs between about 72 hours and about 168 hours. In some aspects, the second temperature set point occurs at about 72 hours, about 78 hours, about 84 hours, about 90 hours, about 96 hours, about 102 hours, about 108 hours, about 114 hours, about 120 hours, about 126 hours, about 132 hours, about 138 hours, about 144 hours, about 150 hours, about 156 hours, about 162 hours, or about 168 hours. In some aspects, the second temperature set point is about 33° C. after about 140 hours.

In some aspects, the conditions improve the protein yield by at least 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, or at least about 400%; as compared to a reference method without the suitable conditions.

In some aspects, the suitable condition further comprises manganese in the bioreactor. In some aspects, the manganese is at a concentration from about 1.6 parts per billion (ppb) to about 15 ppb. In some aspects, the manganese is at a concentration of about 3 ppb to about 6 ppb.

In some aspects, the method reduces cell growth rate. In some aspects, the method controls a cell viability. In some aspects, the cell viability exhibits a mean peak viable cell density (VCD) between about 10.0×10⁶ cells/mL and about 15.0×10⁶ cells/mL. In some aspects, the method controls a titer. In some aspects, the titer exhibits a final titer between about 1.50 g/L and about 3.5 g/L. In some aspects, the titer exhibits a final titer great than about 2.00 g/L.

In some aspects, the method controls a glycosylation profile of the protein. In some aspects, the glycosylation profile comprises one or more N-linked glycans. In some aspects, the glycosylation profile is measured during the protein production phase. In some aspects, the glycosylation profile is measured about every 1 day. In some aspects, the glycosylation profile is measured when the cell culture is harvested. In some aspects, the N-linked glycans comprise: G0F, G1F, G2F, S1G1F, S1G2F, S2G2F, or any combination thereof. In some aspects, the protein comprises a CTLA4 domain. In some aspects, the protein is a fusion protein. In some aspects, the fusion protein comprises an Fc portion. In some aspects, the protein is belatacept. In some aspects, the protein comprises an amino acid sequence at least about 80%, at least about 85%, at least about 90%, at least about 95%, at least about 96%, at least about 97%, at least about 98%, at least about 99%, or about 100% identical to SEQ ID NO: 5. In some aspects, the one or more N-linked glycans are located at one or more residues selected from the group consisting of Asn76, Asn108, and/or Asn207 of belatacept. In some aspects, the one or more N-linked glycans comprises sialic acid and have a molar ratio of NANA of from about 4 to about 10. In some aspects, the one or more N-linked glycans comprises sialic acid and have a molar ratio of NANA of from about 5 to about 9, from about 5.5 to about 8.5, from about 5.8 to about 6.7, from about 5.2 to about 7.5, from about 6 to about 8, from about 6.2 to about 7.4, or from about 5 to about 6. In some aspects, the molar ratio of NANA is about 6.8. In some aspects, the glycosylation profile is analyzed via a N-linked carbohydrate profile method. In some aspects, the glycosylation profile includes one or more O-linked glycans. In some aspects, the O-linked glycans are located at residues Ser129, Ser130, Ser136, and/or Ser139.

In some aspects, the method is performed as a fed-batch culture process. In some aspects, glucose and/or galactose are supplemented to a feed media in the bioreactor. In some aspects, the feed media is added to the bioreactor periodically. In some aspects, the feed media is added to the bioreactor about every 24 hours.

In some aspects, the method is performed as a perfusion process. In some aspects the cells are mammalian cells. In some aspects, the mammalian cells are Chinese hamster ovary (CHO) cells. In some aspects, the mammalian cells are CHO-K1 cells, CHO-DXB11 cells, or CHO-DG44 cells.

The present disclosure is also related to a method of analyzing glycans of a CTLA4-Fc fusion protein, comprising measuring one or more N-linked glycans attached to one or more asparagine residues in the CTLA4 protein, wherein one of the glycans comprises G0F, G1F, G2F, S1G1F, S1G2F, and/or S2G2F. In some aspects, the glycans are measured via Ultra Performance Liquid Chromatography with fluorescence detection (UPLC-FLR). In some aspects, the Fc domain of the CTLA4-Fc fusion protein is cleaved prior to the measuring. In some aspects, the Fc domain of the CTLA4-Fc fusion protein is not cleaved prior to the measuring.

In some aspects, a protein is produced by the methods described herein. In some aspects, the protein comprises a CTLA4-Fc fusion protein. In some aspects, the protein is belatacept. In some aspects, a cell is produced by the methods described herein. In some aspects, the cell is a mammalian cell. In some aspects, the cell is a Chinese hamster ovary (CHO) cell. In some aspects, the cell is a CHO-K1 cell, CHO-DXB11 cell, or CHO-DG44 cell.

The present disclosure also relates to a bioreactor for the manufacture of a protein produced by the methods described herein. In some aspects, the bioreactor comprises the cells described herein and a cell culture medium, wherein the bioreactor is maintained at a pH of about 7.15. In some aspects, the bioreactor is maintained at: (a) an initial temperature set point of about 36° C., a second temperature set point of about 33° C., and a third temperature set point of about 31° C.; (b) a pH set point of about 7.15; and (c) an initial viable cell density (VCD) set point of about 0.70×10⁶ cells/mL. In some aspects, the cell culture medium further comprises manganese.

### BRIEF DESCRIPTION OF THE DRAWINGS

FIG. 1A shows the operating parameters of three processes, Process A, Process B, and Process X. FIGS. 1B and 1C show additional process parameters for Process A, Process B, Process X.

FIG. 2 shows the measured sialic acid (NANA) molar ratio concent of the cell culture during upstream production over 16 days of culture.

FIGS. 3A and 3B show full-scale and zoomed representative chromatograms of the N-glycan profiling of a Belatacept Reference Standard (RS) elution, with specific glycans labeled (G0F, G1F, G2F, S1G1F, and S2G2F).

FIGS. 4A and 4B show a full-scale and zoomed representative chromatogram of the N-glycan profiling of a Belatacept RS elution, where glycan S1G1F (“S1G1F_2”) elutes as two peaks.

FIG. 5 shows an overview of the upstream production process for Belatacept.

FIGS. 6A and 6B show exemplary structures of G0F (mannose-3-N-acetylglucosamine-4-fucose), G1F (mannose-3-N-acetylglucosamine-4-galactose-1-fucose), G2F (mannose-3-N-acetylglucosamine-4-galactose-2-fucose), S1G1F (mono-sialylated mannose-3-N-acetylglucosamine-4-galactose-1-fucose), S1G2F (mono-sialylated mannose-3-N-acetylglucosamine-4-galactose-2-fucose), S1G3F (mono-sialylated mannose-3-N-acetylglucosamine-4-galactose-3-fucose), and S2G2F (di-sialylated mannose-3-N-acetylglucosamine-4-galactose-2-fucose).

### DETAILED DESCRIPTION OF THE DISCLOSURE

The present disclosure is directed to methods of improving the yield of a protein in cells while maintaining the desired property, e.g., glycosylation pattern, of the protein. In particular, the disclosure shows a surprising effect of a pH set point during the production of a protein, especially in the protein induction phase, in a bioreactor. In some aspects, the methods of improving yields comprises culturing the cells in a bioreactor for a protein induction phase under suitable conditions including, but not limited to, adjusting to temperatures, setting pH, using a particular viable cell density, or any combination thereof.

### I. Definitions

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

As used herein, the term “CTLA4 extracellular domain” refers to a protein domain comprising all or a portion of the amino acid sequence shown in SEQ ID NO: 1, that binds to B7-1 (CD80) and/or B7-2 (CD86). In some aspects, a CTLA4 extracellular domain can comprise a polypeptide having an amino acid sequence that is at least 75%, 80%, 85%, 90%, 95%, 96%, 97%, 98%, or 99% identical to SEQ ID NO: 1. A CTLA4 extracellular domain is represented by the following sequence:

[CTLA4 Extracellular Domain]
SEQ ID NO: 1
MHVAQPAVVLASSRGIASFVCEYASPGKATEVRVTVLRQADSQVTEVCA
ATYMMGNELTFLDDSICTGTSSGNQVNLTIQGLRAMDTGLYICKVELMY
PPPYYLGIGNGTQIYVIDPEPCPDSD

As used herein, the terms “CTLA4-Ig” or “CTLA4-Ig molecule” or “CTLA4Ig molecule” or “CTLA4-Ig protein” or “CTLA4Ig protein” or “CTLA4-Fc” are used interchangeably, and refer to a protein molecule that comprises at least a CTLA4-Ig polypeptide having a CTLA4 extracellular domain and an immunoglobulin constant region or portion thereof. In some aspects, for example, a CTLA4-Ig polypeptide comprises at least the amino acid sequence of SEQ ID NO: 2. In certain aspects, the CTLA4 extracellular domain and the immunoglobulin constant region or portion thereof can be wild-type, or mutant or modified. A mutant CTLA4-Ig polypeptide is a CTLA4-Ig polypeptide comprising a mutant CTLA4 extracellular domain. A mutant CTLA4Ig molecule comprises at least a mutant CTLA4-Ig polypeptide. In some aspects, the CTLA4 extracellular domain and the immunoglobulin constant region or portion thereof can be mammalian, including human or mouse. In some aspects, a mutant CTLA4 extracellular domain can have an amino acid sequence that is at least 75%, 80%, 85%, 90%, 95%, 96%, 97%, 98%, or 99% identical to the CTLA4 extracellular domain shown in any one or more of SEQ ID NO: 2, 3, 4, 5, 6, 7, or 8. The polypeptide can further comprise additional protein domains. A CTLA4-Ig molecule can refer to a monomer of the CTLA4-Ig polypeptide, and can refer to multimer forms of the polypeptide, such as dimers, tetramers, and hexamers, etc. (or other high molecular weight species). CTLA4-Ig molecules are also capable of binding to CD80 and/or CD86. Examples of CTLA4-Ig and fragments (e.g., belatacept) are shown in SEQ ID NOs: 2, 3, 4, 5, 6, 7, and 8. In some aspects, belatacept is a combination of SEQ ID NO: 2, 3, 4, 5, 6, 7, and 8.

[CTLA4^{A29YL104E}-Ig amino acid sequence]
SEQ ID NO: 2
MGVLLTQRTLLSLVLALLFPSMASMAMHVAQPAVVLASSRGIASFVCEYASPGKYTEVRV
TVLRQADSQVTEVCAATYMMGNELTFLDDSICTGTSSGNQVNLTIQGLRAMDTGLYICKV
ELMYPPPYYEGIGNGTQIYVIDPEPCPDSDQEPKSSDKTHTSPPSPAPELLGGSSVFLF
PPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVF
SCSVMHEALHNHYTQKSLSLSPGK
[amino acids 25-383 of SEQ ID NO: 2]
SEQ ID NO: 3
MAMHVAQPAVVLASSRGIASFVCEYASPGKYTEVRVTVLRQADSQVTEVCAATYMMGNELTFLDD
SICTGTSSGNQVNLTIQGLRAMDTGLYICKVELMYPPPYYEGIGNGTQIYVIDPEPCPDSDQEPK
SSDKTHTSPPSPAPELLGGSSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
[amino acids 26-383 of SEQ ID NO: 2]
SEQ ID NO: 4
AMHVAQPAVVLASSRGIASFVCEYASPGKYTEVRVTVLRQADSQVTEVCAATYMMGNELTFLDDS
ICTGTSSGNQVNLTIQGLRAMDTGLYICKVELMYPPPYYEGIGNGTQIYVIDPEPCPDSDQEPKS
SDKTHTSPPSPAPELLGGSSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGK
[amino acids 27-383 of SEQ ID NO: 2]
SEQ ID NO: 5
MHVAQPAVVLASSRGIASFVCEYASPGKYTEVRVTVLRQADSQVTEVCAATYMMGNELTFLDDSI
CTGTSSGNQVNLTIQGLRAMDTGLYICKVELMYPPPYYEGIGNGTQIYVIDPEPCPDSDQEPKSS
DKTHTSPPSPAPELLGGSSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDKS
RWQQGNVFSCSVMHEALHNHYTQKSLSLSPGK
[amino acids 25-382 of SEQ ID NO: 2]
SEQ ID NO: 6
MAMHVAQPAVVLASSRGIASFVCEYASPGKYTEVRVTVLRQADSQVTEVCAATYMMGNELTFLDD
SICTGTSSGNQVNLTIQGLRAMDTGLYICKVELMYPPPYYEGIGNGTQIYVIDPEPCPDSDQEPK
SSDKTHTSPPSPAPELLGGSSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPG
[amino acids 26-382 of SEQ ID NO: 2]
SEQ ID NO: 7
AMHVAQPAVVLASSRGIASFVCEYASPGKYTEVRVTVLRQADSQVTEVCAATYMMGNELTFLDDS
ICTGTSSGNQVNLTIQGLRAMDTGLYICKVELMYPPPYYEGIGNGTQIYVIDPEPCPDSDQEPKS
SDKTHTSPPSPAPELLGGSSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDK
SRWQQGNVFSCSVMHEALHNHYTQKSLSLSPG
[amino acids 27-382 of SEQ ID NO: 2]
SEQ ID NO: 8
CTGTSSGNQVNLTIQGLRAMDTGLYICKVELMYPPPYYEGIGNGTQIYVIDPEPCPDSDQEPKSS
DKTHTSPPSPAPELLGGSSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDSDGSFFLYSKLTVDKS
RWQQGNVFSCSVMHEALHNHYTQKSLSLSPG
[CTLA4 Extracellular Domain with A29Y and L104E Mutations]
SEQ ID NO: 9
MHVAQPAVVLASSRGIASFVCEYASPGKYTEVRVTVLRQADSQVTEVCAATYMMGNELTFLDDSI
CTGTSSGNQVNLTIQGLRAMDTGLYICKVELMYPPPYYEGIGNGTQIYVIDPEPCPDSD

As used herein, the term “soluble CTLA4” means a molecule that can circulate in vivo or CTLA4 which is not bound to a cell membrane. For example, the soluble CTLA4 can include CTLA4-Ig which includes the extracellular region of CTLA4, linked to an Ig.

As used herein, the term “dimer” refers to a CTLA4-Ig protein or CTLA4-Ig molecule composed of two CTLA4-Ig polypeptides or monomers linked or joined together. The linkage between monomers or a dimer can be a non-covalent linkage or interaction, a covalent linkage or interaction (e.g., one or more disulfide bonds), or both. A CTLA4-Ig protein or CTLA4-Ig molecule composed of two identical monomers is a homodimer. A CTLA4-Ig homodimer also encompasses a molecule comprising two monomers that can differ slightly in sequence. A homodimer encompasses a dimer where the monomers joined together have substantially the same sequence. The monomers comprising a homodimer share considerable structural homology. For example, the differences in sequence can be due to N-terminal processing modifications of the monomer.

As used herein, the terms “glutamate” and “glutamic acid” are used interchangeably.

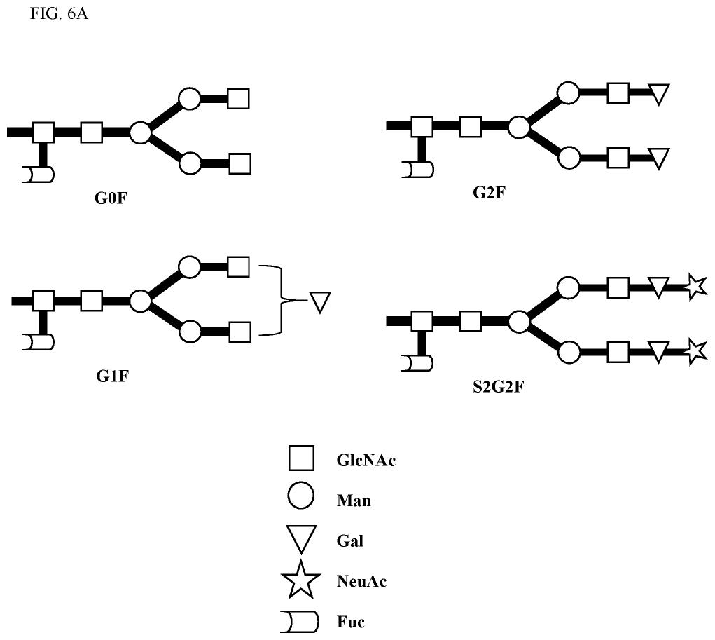

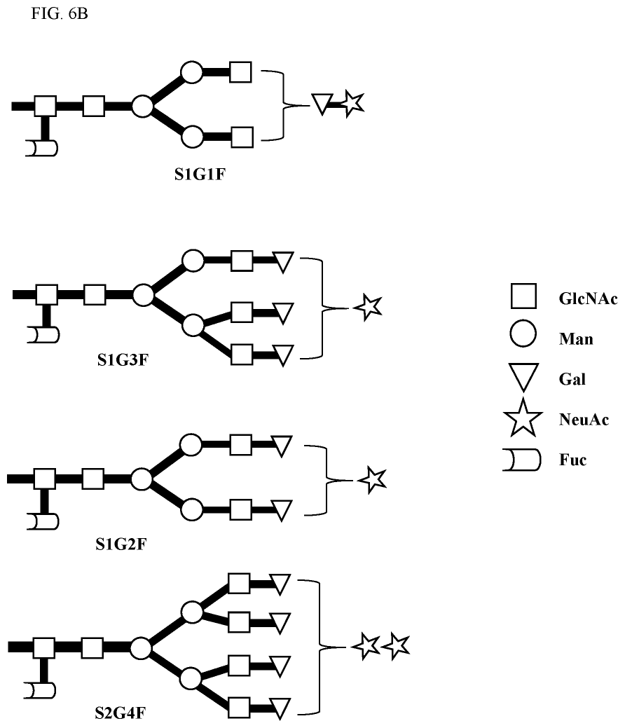

As used herein, Asn76, Asn108 (CTLA4 region) and Asn207 (Fc region) refer to specific glycosylation sites present on the Belatacept molecule. These labels correspond to Asparagine 76, Asparagine 108, and Asparagine, respectively, and correspond to the residues in SEQ ID NO: 5. Generally, a five-character code is used to define carbohydrate structural classes, e.g., P2100, P2120, P2121, P3131, and P4142. With respect to this labeling scheme, the first character (P) in the code defines the released carbohydrate as an N-linked structure containing a trimannosyl core structure. The second character represents the number of N-Acetylglucosamine (GlcNAc) units attached to the core. The third character (0 or 1) represents whether there is a fucose (Fuc) attached to the first GlcNAc of the core. The fourth character represents the number of Galactose (Gal) sugars attached to the core. The fifth character represents the number of SA (N-acetylneuraminic acid or N-glycolylneuraminic acid) attached to the carbohydrate. P2100 can be also denoted as G0F. P2110 can be also denoted as G1F. P2120 can be also denoted as G2F. P2121 can be also denoted as S1G2F. P2122 can be also denoted as S2G2F. P3131 can be also denoted as S1G3F. P4142 can be also denoted as S2G4F. Representative diagrams of these glycans can be seen in FIGS. 6A and 6B.

As used herein, the term “purified” refers to a composition comprising a protein, e.g., CTLA4-Ig molecule, or a selected population of proteins, e.g., CTLA4-Ig molecules, that is removed from its natural environment (e.g., is isolated) and is at least 90% free, 91% free, 92% free, 93% free, 94% free, 95% free, 96% free, 97% free, 98% free, 99% free, 99.5% free, or 99.9% free from other components, such as cellular material or culture medium, with which it is naturally associated. “Purified” can also refer to a composition comprising a protein, e.g., CTLA4-Ig molecule, or a selected population of proteins, e.g., CTLA4-Ig molecules, that is removed from its natural environment and is at least 60% free, 65% free, 70% free, 75% free, 80% free, or 85% free from other components, such as cellular material or culture medium, with which it is naturally associated. For example, with respect to a recombinantly produced protein, e.g., CTLA4-Ig protein molecule, the term “purified” can also refer to a composition comprising a protein, e.g., CTLA4-Ig protein molecule that is removed from the production environment such that the protein molecule is at least 90% free, 91% free, 92% free, 93% free, 94% free, 95% free, 96% free, 97% free, 98% free, 99% free, 99.5% free, or 99.9% free from protein molecules which are not polypeptides of SEQ ID NO: 2 or mutant polypeptides of SEQ ID NO: 2 which are of interest. “Purified” does not exclude mixtures of proteins, e.g., CTLA4-Ig molecules (such as dimers) with other variant proteins, e.g., CTLA4-Ig molecules (such as tetramer). “Purified” does not exclude pharmaceutically acceptable excipients or carriers combined with the proteins, e.g., CTLA4-Ig molecules, wherein the proteins, e.g., CTLA4-Ig molecules, have been taken out of their native environment.

As used herein, the term “large-scale process” is used interchangeably with the term “industrial-scale process”. The term “culture vessel” is used interchangeably with “bioreactor”, “reactor” and “tank”. A bioreactor used on an industrial scale can be at least 2,000 L, at least 5,000 L, at least 10,000 L, at least 15,000 L, at least 20,000 L, at least 25,000 L, or any size appropriate for the large production scale necessary to produce an industrial supply.

A “liquid culture” refers to cells (for example, bacteria, plant, insect, yeast, or animal cells) grown on supports, or growing suspended in a liquid nutrient medium.

A “seed culture” refers to a cell culture grown in order to be used to inoculate larger volumes of culture medium. The seed culture can be used to inoculate larger volumes of media in order to expand the number of cells growing in the culture (for example, cells grown in suspension).

As used herein, the terms “culture medium” and “cell culture medium” and “feed medium” and “fermentation medium” refer to a nutrient solutions used for growing and or maintaining cells, especially mammalian cells. Without limitation, these solutions ordinarily provide at least one component from one or more of the following categories: (1) an energy source, usually in the form of a carbohydrate such as glucose; (2) all essential amino acids, and usually the basic set of twenty amino acids plus cysteine; (3) vitamins and/or other organic compounds required at low concentrations; (4) free fatty acids or lipids, for example linoleic acid; and (5) trace elements, where trace elements are defined as inorganic compounds or naturally occurring elements that are typically required at very low concentrations, usually in the micromolar range. The nutrient solution can be supplemented electively with one or more components from any of the following categories: (1) hormones and other growth factors such as, serum, insulin, transferrin, and epidermal growth factor; (2) salts, for example, magnesium, calcium, and phosphate; (3) buffers, such as HEPES; (4) nucleosides and bases such as, adenosine, thymidine, and hypoxanthine; (5) protein and tissue hydrolysates, for example peptone or peptone mixtures which can be obtained from purified gelatin, plant material, or animal byproducts; (6) antibiotics, such as gentamycin; (7) cell protective agents, for example pluronic polyol; and (8) galactose. Commercially available media such as Ham's F10 (Sigma), Minimal Essential Medium ((MEM), (Sigma)), RPMI-1640 (Sigma), and Dulbecco's Modified Eagle's Medium ((DMEM), (Sigma)) are suitable for culturing the host cells. In addition, any of the media described in Ham et al., Meth. Enz. 58:44 (1979), Barnes et al., Anal. Biochem. 102:255 (1980) can be used as culture media for the host cells. Any other necessary supplements can also be included at appropriate concentrations.

As used herein, “culturing” refers to growing one or more cells in vitro under defined or controlled conditions. Examples of culturing conditions which can be defined include temperature, gas mixture, time, and medium formulation.

As used herein, “expanding” refers to culturing one or more cells in vitro for the purpose of obtaining a larger number of cells in the culture.

As used herein, “temperature set point” refers to the temperature setting of a bioreactor or other upstream processing vessel used to grow cells and/or produce protein product. A temperature set point can be established at the outset of cell culture, where it can also be referred to as an “initial temperature set point”. Subsequent changes in temperature during cell culture after the initial temperature set point can be referred to using ordinal numbering, i.e., a second temperature set point, or subsequently a third temperature set point. The last temperature set point before downstream processing can also be referred to as a “final temperature set point”. In some instances, a process can comprise an initial temperature set point, a second temperature set point, and third (and final) temperature set point.

As used herein, “set point” refers to the initial setting of the condition in a bioreactor or other upstream processing vessel used to grow cells and/or produce protein product unless otherwise indicated. A set point is established at the outset of cell culture. Subsequent changes in the condition during cell culture after the set point can occur due to variations in cell culture media conditions during growth. For example, a set point can be a pH set point. In some aspects, a set point is a temperature set point. In some aspects, the set point can be maintained throughout the cell culturing method. In other aspects, the set point can be maintained until a different set point is set. In other aspects, the set point can be changed to another set point.

As used herein, “population” refers to a group of two or more molecules (“population of molecules”) or cells (“population of cells”) that are characterized by the presence or absence of one or more measurable or detectable properties. In a homogeneous population, the molecules or cells in the population are characterized by the same or substantially the same properties (for example, the cells of a clonal cell line). In a heterogeneous population, the molecules or cells in the population are characterized by at least one property that is the same or substantially the same, where the cells or molecules can also exhibit properties that are not the same (for example, a population of proteins, CTLA4-Ig molecules, having a substantially similar average sialic content, but having non-similar mannose content).

As used herein, “high molecular weight aggregate” is used interchangeably with “high molecular weight species” or “HMW” to refer to a CTLA4-Ig molecule comprising at least three CTLA4-Ig monomers. For example, a high molecular weight aggregate can be a tetramer, a pentamer or a hexamer.

As used herein “Protein A” refers to a protein that is approximately 42 kDa and binds very strongly to the Fc portion of an immunoglobulin, and its use in the purification of antibodies is well known in the art. Protein A has been extensively used in the art for purification. (Boyle et al., 1993; Hou et al. 1991). When applied to Protein A, the term “residual”, or “rPA” refers to any remaining Protein A present in a mixture due its use in purification of a protein of interest or an antibody further upstream in a manufacturing process.

As used herein, “protein yield” or “yield” refers to the total amount of protein of interest harvested from a culture process, i.e., a bioreactor cell culture process. “Protein yield” or “yield” can also refer to the total amount of protein of interest recovered after the processes disclosed herein, and can be measured by mass (i.e., grams).

As used herein, “titer” or “protein titer” refers to the concentration of a protein of interest in a solution. For example, a measurement of the concentration of a protein of interest in an upstream culture process, i.e., a bioreactor, represents the protein titer at that moment in time. Titer can be measured in a concentration format (e.g., mg/ml) in a fixed volume.

As used herein, “glycosylation content” refers to an amount of N-linked or O-linked sugar residues covalently attached to a protein molecule, such as a glycoprotein like a CTLA4-Ig molecule.

As used herein, “glycosylation” refers to the addition of complex oligosaccharide structures to a protein at specific sites within the polypeptide chain. Glycosylation of proteins and the subsequent processing of the added carbohydrates can affect protein folding and structure, protein stability, including protein half-life, and functional properties of a protein. Protein glycosylation can be divided into two classes by virtue of the sequence context where the modification occurs, O-linked glycosylation and N-linked glycosylation. O-linked polysaccharides are linked to a hydroxyl group, usually to the hydroxyl group of either a serine or a threonine residue. O-glycans are not added to every serine and threonine residue. O-linked oligosaccharides are usually mono or biantennary, i.e. they comprise one or at most two branches (antennas), and comprise from one to four different kinds of sugar residues, which are added one by one. N-linked polysaccharides are attached to the amide nitrogen of an asparagine. Only asparagines that are part of one of two tripeptide sequences, either asparagine-X-serine or asparagine-X-threonine (where X is any amino acid except proline), are targets for glycosylation. N-linked oligosaccharides can have from one to four branches referred to as mono-, bi-, tri-tetraantennary. The structures of and sugar residues found in N- and O-linked oligosaccharides are different. Despite that difference, the terminal residue on each branch of both N- and O-linked polysaccharide can be modified by a sialic acid molecule a modification referred as sialic acids capping. Sialic acid is a common name for a family of unique nine-carbon monosaccharides, which can be linked to other oligosaccharides. Two family members are N-acetyl neuraminic acid, abbreviated as Neu5Ac, NeuAc, or NANA, and N-glycolyl neuraminic acid, abbreviated as Neu5Gc or NGNA. The most common form of sialic acid in humans is NANA. N-acetylneuraminic acid (NANA) is the primary sialic acid species present in CTLA4-Ig molecules. However, it should be noted that minor but detectable levels of N glycolylneuraminic acid (NGNA) are also present in CTLA4-Ig molecules. Furthermore, the method described herein can be used to determine the number of moles of sialic acids for both NANA and NGNA, and therefore levels of both NANA and NGNA are determined and reported for CTLA4-Ig molecules. N- and O-linked oligosaccharides have different number of branches, which provide different number of positions to which sialic acid molecules can be attached. N-linked oligosaccharides can provide up to four attachment positions for sialic acids, while 0-linked oligosaccharides can provide two sites for sialic acid attachment.

As used herein, the term “molar ratio of sialic acids to protein” or “MR” is calculated and given as number of moles of sialic acid molecules per moles of protein (CTLA4-Ig molecules) or dimer.

As used herein, the term “glycoprotein” refers to a protein that is modified by the addition of one or more carbohydrates, including the addition of one or more sugar residues.

As used herein, the term “sialylation” refers to the addition of a sialic acid residue to a protein, including a glycoprotein.

As used herein, the term “glycoprotein isoform” refers to a molecule characterized by its carbohydrate and sialic acid content as determined by isoelectric focusing (IEF) gel electrophoresis or other suitable methods for distinguishing different proteins in a mixture by their molecular weight, charge, and/or other characteristics. For example, each distinct band observed on an IEF gel represents molecules that have a particular isoelectric point (pI) and thus the same net overall charge. A glycoprotein isoform can be a distinct band observed on an IEF gel where each band can be a population of molecules that have a particular pI.

As used herein, the term “beta polypeptide” refers to a mutant CTLA4-Ig polypeptide that (1) comprises the amino acid sequence of SEQ ID NO: 1 wherein the amino acid at position 29 is mutated to tyrosine and the amino acid at position 104 is mutated to glutamate, optionally with various additional mutations, and an immunoglobulin constant region, or a portion thereof, and (2) is capable of binding to CD80 and/or CD86. In some aspects, for example, a beta polypeptide comprises at least the amino acid sequence of the extracellular domain of CTLA4^{A29YL104E}-Ig (as shown in SEQ ID NO: 9). Non-limiting examples of beta polypeptides include belatacept and SEQ ID NOS: 2-8. In certain aspects, the immunoglobulin constant region or portion thereof can be wild-type, or mutant or modified. In certain aspects, the immunoglobulin constant region or portion thereof can be mammalian, including human or mouse. Additional non-limiting examples of beta polypeptides include a beta polypeptide comprising one or more amino acid mutations in the immunoglobulin constant region or portion thereof (for example, substitution of cysteine 120 of SEQ ID NO: 2), and a beta polypeptide comprising further mutations at one or more of amino acid position 25, 30, 93, 96, 103 or 105 of SEQ ID NO: 1. A beta polypeptide molecule comprises a beta polypeptide. A beta polypeptide molecule can refer to a monomer of the beta polypeptide and multimer forms of the beta polypeptide, such as dimers, tetramers and hexamers, etc. For example, belatacept comprises beta polypeptide molecules. Beta polypeptide molecules are further described in U.S. Pat. No. 10,508,144, issued on Dec. 17, 2019, which is hereby incorporated by reference in its entirety.

“Potency” refers to a measure of the response as a function of ligand concentration. For example, agonist potency is quantified as the concentration of ligand that produces half the maximal effect (EC₅₀). A non-limiting pharmacological definition of potency includes components of affinity and efficacy, where, efficacy is the ability of a drug to evoke a response once bound. Potency is related to affinity, but potency and affinity are different measures of drug action.

As used herein, “pharmaceutically acceptable carrier” refers to a vehicle for a pharmacologically active agent. The carrier facilitates delivery of the active agent to the target site without terminating the function of the agent. Non-limiting examples of suitable forms of the carrier include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration.

As used herein, the phrase “pharmaceutically acceptable composition” (or “pharmaceutical composition”) refers to a composition that is acceptable for pharmaceutical administration, such as to a human being. Such a composition can include substances that are impurities at a level not exceeding an acceptable level for pharmaceutical administration (such level including an absence of such impurities), and can include pharmaceutically acceptable excipients, vehicles, carriers and other inactive ingredients, for example, to formulate such composition for ease of administration, in addition to any active agent(s). For example, a pharmaceutically acceptable CTLA4-Ig composition can include MCP-1 or DNA, so long as those substances are at a level acceptable for administration to humans.

“Drug substance” is the active pharmaceutical ingredient contained in a pharmaceutical composition. The term “drug substance” includes an active pharmaceutical ingredient in solution and/or in buffered form. “Drug product” is a pharmaceutical composition containing drug substance formulated for pharmaceutical administration. For purposes of the assays contained in the Examples and elsewhere herein, which can refer to drug substance and/or drug product, exemplary drug substances and drug products that can be assayed are as follows.

Exemplary drug product for CTLA4Ig molecules include:

Composition of lyophilized CTLA4-Ig
protein (250 mg/vial) drug product

| Component | Amount (mg/vial) |
|---|---|
| CTLA4-Ig protein | 262.5 |
| Maltose monohydrate | 525 |
| Sodium phosphate monobasic, monohydrate | 18.1 |
| Sodium chloride | 15.3 |
| Hydrochloric Acid | Adjust to pH 7.5 |
| Sodium hydroxide | Adjust to pH 7.5 |

The term “inoculation” as used herein refers to the addition of cells to culture medium to start the culture.

The term “induction” or “induction phase” or “growth phase” of the cell culture as used herein refers to the initial seeding of the bioreactor at the outset of upstream cell culture, and includes the period of exponential cell growth (for example, the log phase) where cells are primarily dividing rapidly. During this phase, the rate of increase in the density of viable cells is higher than at any other time point.

As used herein, the term “production phase” of the cell culture refers to the period of time during which cell growth is stationary or is maintained at a near constant level. The density of viable cells remains approximately constant over a given period of time. Logarithmic cell growth has terminated and protein production is the primary activity during the production phase. The medium at this time is generally supplemented to support continued protein production and to achieve the desired glycoprotein product.

As used herein, the terms “expression” or “expresses” are used to refer to transcription and translation occurring within a cell. The level of expression of a product gene in a host cell can be determined on the basis of either the amount of corresponding mRNA that is present in the cell or the amount of the protein encoded by the product gene that is produced by the cell, or both.

As used herein “N-linked glycan” refers to a protein modification where a glycan is linked to a glycoconjugate via a nitrogen linkage. The acceptors of the glycan are selected asparagine residues of polypeptide chains that have entered the periplasm or the lumen of the ER, respectively. Oligosaccharyltransferase, the central enzyme of the N-glycosylation pathway, catalyzes the formation of an N-glycosidic linkage of the oligosaccharide to the side-chain amide of asparagine residues that are specified by the consensus sequence N-X-S/T. All eukaryotic N-glycans share a common core sequence, Manα1-3(Manα1-6)Manβ1-4GlcNAcβ1-4GlcNAcβ1-Asn-X-Ser/Thr, and are classified into three types: (1) oligomannose, in which only Man residues extend the core; (2) complex, in which “antennae” initiated by GlcNAc extend the core; and (3) hybrid, in which Man extends the Manα1-6 arm of the core and one or two GlcNAcs extend the Manα1-3 arm.

As used herein, “N-linked glycosylation” refers the attachment of oligosaccharides to a nitrogen atom, usually the N4 of asparagine residues. N-glycosylation can occur on secreted or membrane bound proteins, mainly in eukaryotes and archaea. A detailed review of the biosynthetic pathways and enzymes used to generate N-linked glycans (e.g., high mannose type oligosaccharides) are described in Stanley et al., “N-Glycans” in Essentials of Glycobiology, Ed. Varki, Cummings, and Eskho, Cold Spring Harbor Press, 2009.

The term “fed-batch”, “fed-batch culture” or “fed-batch culture process” as used herein refers to a method of culturing cells in which additional components are provided to the culture at some time subsequent to the beginning of the culture process. A fed-batch culture can be started using a basal medium. The culture medium with which additional components are provided to the culture at some time subsequent to the beginning of the culture process is a feed medium. A fed-batch culture is typically stopped at some point and the cells and/or components in the medium are harvested and optionally purified.

As used herein “perfusion” or “perfusion culture” or “perfusion culture process” refers to continuous flow of a physiological nutrient solution at a steady rate, through or over a population of cells. As perfusion systems generally involve the retention of the cells within the culture unit, perfusion cultures characteristically have relatively high cell densities, but the culture conditions are difficult to maintain and control. In addition, since the cells are grown to and then retained within the culture unit at high densities, the growth rate typically continuously decreases over time, leading to the late exponential or even stationary phase of cell growth. This continuous culture strategy generally comprises culturing mammalian cells, e.g., non-anchorage dependent cells, expressing a polypeptide and/or virus of interest during a production phase in a continuous cell culture system.

As used herein, the term “mass spectrometry” refers to a sensitive technique used to detect, identify and quantitate molecules based on their mass-to-charge (m/z) ratio. Electric fields are used to separate ions according to their mass-to-charge ratio (m/z), the ratio of mass to the integer number of charges (z), as they pass along the central axis of parallel and equidistant poles or rods, such as a quadrupole, which contains four poles or rods. Each rod has two voltages applied, one of which is a fixed direct current and the second is an alternating current that cycles with a superimposed radio frequency. The magnitude of the applied electric field can be ordered such that only ions with a specific m/z ratio can travel through the quadrupole, prior to being detected. Ions with all other m/z values are deflected onto trajectories that would cause them to collide with the quadrupole rods and discharge, or be ejected from the mass analyzer field and removed via the vacuum. The quadrupole is often referred to as an exclusive detector because only ions with a specific m/z are stable in the quadrupole at any one time. Those ions with a stable trajectory are often referred to as having noncollisional, resonant or stable trajectories.

Generally, for an experiment on a triple-quadrupole mass spectrometer, the first quadrupole (Q1) is set to pass ions only of a specified m/z (precursor ions) of an expected chemical species in the sample. The second quadrupole (i.e. Q2 or the collision cell) is used to fragment the ions passing through Q1. The third quadrupole (Q3) is set to pass to the detector only ions of a specified m/z (fragment ions) corresponding to an expected fragmentation product of the expected chemical species. In some aspects, the sample is ionized in the mass spectrometer to generate one or more protonated or deprotonated molecular ions. In some aspects, the one or more protonated or deprotonated molecular are singly charged, doubly charged, triply charged or higher. In some aspects, the mass spectrometer is a triple quadrupole mass spectrometer. In some aspects, the resolutions used for Q1 and Q3 are unit resolution. In other aspects, the resolutions used for Q1 and Q3 are different. In other aspects, the resolution used for Q1 is higher than the unit resolution of Q3.

As used herein, the term “fluorophore” refers to a fluorescent chemical compound that can re-emit light upon light excitation. Fluorophores typically contain several combined aromatic groups, or planar or cyclic molecules with several π bonds. Two commonly used fluorophores are 2-AB (2-aminobenzamide) and 2-AA (anthranilic acid or 2-aminobenzoic acid). Other fluorophores include PA (2-Aminopyridine), AMAC (2-aminoacridone), ANDS (7-Amino-1,3-naphthalenedisulfonic acid), ANTS (8-Aminonaphthalene-1,3,6-trisulfonic acid), APTS (9-Aminopyrene-1,4,6-trisulfonic acid), and 3-(acetylamino)-6-aminoacridine.

A “glycan profile” as used in the disclosure should be understood to be any defined set of values of quantitative results for glycans that can be used for comparison to reference values or profiles derived from another sample or a group of samples. For instance, a glycan profile of a sample from a protein sample might be significantly different from a glycan profile of a sample from an alternate source. A glycan profile can aid in predicting or anticipating a protein's pharmacodynamic (PD) or pharmacokinetic (PK) therapeutic effects by comparing the profile to a reference or standard profile. Reference and sample glycan profiles can be generated by any analysis instrument capable of detecting glycans, such as mass spectrometry. The one or more N-glycans can be Galactose (Gal), N-Acetylgalactosamine (GalNAc), Galactosamine (GalN), Glucose (Glc), N-Acetylglucosamine (GlcNAc), Glucosamine (GlcN), Mannose (Man), N-Acetylmannosamine (ManNAc), Mannosamine (ManN), Xylose (Xyl), N-Acetylneuraminic acid (Neu5Ac), N-Glycolylneuraminic acid (Neu5Gc), 2-Keto-3-deoxynononic acid (Kdn), Fucose (Fuc), Glucuronic Acid (GlcA), Iduronic acid (IdoA), Galacturonic acid (GalA), Mannuronic acid (ManA), or any combination thereof.

As used herein, the phrase “working solution(s)” refers to solutions that are used in a method. Non-limiting examples of working solutions include buffers.

As used herein, “reference material” refers to a material that is used as a standard in a method. For example, a reference material can be used as a standard to which experimental samples will be compared.

As used herein, “reference method” or “reference process” refers to a process or method of producing the same protein using the same methods except the conditions used for the Process X. For example, a reference process or method can be a process described in Example 1 (Process A), Example 1B (Process B), and/or Example 1C (Process C). As used herein, a reference method can be used as a baseline to which the methods of the present disclosure are compared.

The absence of a substance is contemplated where no lower limit is provided with regard to a range of amounts of such substance.

As used herein, recited temperatures in reference to cell culture refers to the temperature setting on the instrument that regulates the temperature of the bioreactor. Of course, the temperature of the liquid culture itself will adopt the temperature set on the instrument regulating the temperature for the bioreactor. Where the temperature refers to a cell culture that is maintained on a shelf in an incubator, the temperature then refers to the shelf temperature of the incubator.

### II. Method of Improving Protein Production

The present disclosure provides method of improving a protein production phase in a cell culturing system, thereby increasing the yield of a protein. The method disclosed herein therefore is capable of increasing cell culture performance, i.e., any desirable increase in the performance of the cell culture as a result of the present method. By way of example, increased cell culture performance includes, but is not limited to, any one or more of the following: increased protein yield; increased protein titer; increased cell specific productivity; increased maximum cell densities; decrease in high molecular weight species; increase in monomeric species; enhanced cell viability; decreased precipitation in culture media and/or CDFM; enhanced overall product quality as determined by, for example, glycosylation profile and size exclusion chromatography; and enhanced overall lot-to-lot consistency. In some aspects, the method comprises culturing cells that are capable of expressing the protein in a bioreactor for a protein induction phase and/or protein production phase under suitable conditions, wherein the suitable conditions include, but are not limited to, adjustment of an initial temperature set point, adjustment of a second temperature set point, adjustment of a final temperature set point, adjustment of a feed time, adjustment of a pH set point, adjustment of an initial cell density, or adjustment of any combination thereof. In some aspects, the present disclosure provided methods of improving the yield of a protein in cells comprising culturing the cells in a bioreactor for a protein induction phase under suitable conditions, wherein the suitable conditions comprise a pH set point of about 7.15.

The methods of the present disclosure are useful to create reactor conditions where the reactor conditions improve protein yield. In some aspects, the conditions improve the protein yield by at least 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, or at least about 400% as compared to a reference method without the suitable conditions, wherein the suitable conditions comprise adjustment of the pH set point, e.g., to about 7.15. In some aspects, the suitable conditions further comprise adjustment of the initial viable cell density, e.g., 0.70×10⁶ cells/mL, and/or adjustment of the initial, the second, and the final temperature set points, e.g., temperature set points of 36° C., 33° C., and 31° C. In some aspects, the conditions improve the protein yield by at least 150%. In some aspects, the conditions improve the protein yield by at least 160%. In some aspects, the conditions improve the protein yield by at least 170%. In some aspects, the conditions improve the protein yield by at least 180%. In some aspects, the conditions improve the protein yield by at least 190%. In some aspects, the conditions improve the protein yield by at least 200%. In some aspects, the conditions improve the protein yield by at least 210%. In some aspects, the conditions improve the protein yield by at least 220%. In some aspects, the conditions improve the protein yield by at least 230%. In some aspects, the conditions improve the protein yield by at least 240%. In some aspects, the conditions improve the protein yield by at least 250%. In some aspects, the conditions improve the protein yield by at least 260%. In some aspects, the conditions improve the protein yield by at least 270%. In some aspects, the conditions improve the protein yield by at least 280%. In some aspects, the conditions improve the protein yield by at least 290%. In some aspects, the conditions improve the protein yield by at least 300%. In some aspects, the conditions improve the protein yield by at least 310%. In some aspects, the conditions improve the protein yield by at least 320%. In some aspects, the conditions improve the protein yield by at least 330%. In some aspects, the conditions improve the protein yield by at least 340%. In some aspects, the conditions improve the protein yield by at least 3 50%. In some aspects, the conditions improve the protein yield by at least 360%. In some aspects, the conditions improve the protein yield by at least 370%. In some aspects, the conditions improve the protein yield by at least 380%. In some aspects, the conditions improve the protein yield by at least 390%. In some aspects, the conditions improve the protein yield by at least 400%.

In some aspects, the present methods improve the protein yield by at least about 2 fold, at least about 3 fold, at least about 4 fold, at least about 5 fold, at least about 6 fold, at least about 7 fold, at least about 8 fold, at least about 9 fold, or at least about 10 fold higher than a reference method without the suitable conditions, e.g., a pH set point of about 7.15.

In some aspects, suitable conditions include adjustment of the pH set point, e.g., to about 7.15, adjustment of the initial viable cell density, e.g., 0.70×10⁶ cells/mL, and/or adjustment of the initial, the second, and the final temperature set points, e.g., temperature set points of 36° C., 33° C., and 31° C., wherein the methods improve the protein yield by about 2 fold to about 10 fold, e.g., about 2 fold to about 5 fold, about 3 fold to about 5 fold, or about 2 fold to about 4 fold. In some aspects, the present methods improve the protein yield by about 2 fold to about 3 fold. In some aspects, the present methods improve the protein yield by about 3 fold to about 4 fold. In some aspects, the present methods improve the protein yield by about 4 fold to about 5 fold. In some aspects, the present methods improve the protein yield by about 5 fold to about 6 fold. In some aspects, the present methods improve the protein yield by about 6 fold to about 7 fold. In some aspects, the present methods improve the protein yield by about 7 fold to about 8 fold. In some aspects, the present methods improve the protein yield by about 8 fold to about 9 fold.

The methods of the present disclosure are also useful to increase total protein output as a fraction of total host cell protein so that a larger protein yield per batch is achieved. In some aspects, the methods of the present disclosure are also useful to increase total protein output with a desired glycosylation pattern based on alterations to the protein's residence time in the Golgi and exposure to glycosylation enzymes. In some aspects, the conditions improve the protein yield of the protein with the desired glycosylation profile by at least 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, or at least about 400%; as compared to a reference method without the suitable conditions, e.g., adjustment of a pH set point, e.g., a pH of about 7.15, adjustment of the initial viable cell density, e.g., 0.70×10⁶ cells/mL, and/or adjustment of the initial, the second, and the final temperature set points, e.g., temperature set points of 36° C., 33° C., and 31° C.

The methods of the present disclosure are also useful for controlling viable cell density or peak viable cell density during protein production. In some aspects, “controlling viable dell density or peak viable cell density” means maintaining viable cell density at certain ranges of cells/mL. In some aspects, the cell viability exhibits a mean peak viable cell density (VCD) between about 5×10⁶ cells/mL to about 21×10⁶ cells/mL. In some aspects, the cell viability exhibits a mean peak viable cell density (VCD) between about 6×10⁶ cells/mL to about 20×10⁶ cells/mL. In some aspects, the cell viability exhibits a mean peak viable cell density (VCD) between about 7×10⁶ cells/mL to about 19×10⁶ cells/mL. In some aspects, the cell viability exhibits a mean peak viable cell density (VCD) between about 8×10⁶ cells/mL to about 18×10⁶ cells/mL. In some aspects, the cell viability exhibits a mean peak viable cell density (VCD) between about 9×10⁶ cells/mL to about 17×10⁶ cells/mL. In some aspects, the cell viability exhibits a mean peak viable cell density (VCD) between about 10×10⁶ cells/mL to about 16×10⁶ cells/mL. In some aspects, the cell viability exhibits a mean peak viable cell density (VCD) between about 10×10⁶ cells/mL to about 15×10⁶ cells/mL. In some aspects, the cell viability exhibits a mean peak viable cell density (VCD) between about 11×10⁶ cells/mL to about 15×10⁶ cells/mL. In some aspects, the cell viability exhibits a mean peak viable cell density (VCD) between about 12×10⁶ cells/mL to about 14×10⁶ cells/mL.

In some aspects, the cell viability exhibits a mean peak viable cell density (VCD) of about 6×10⁶ cells/mL. In some aspects, the cell viability exhibits a mean peak viable cell density (VCD) of about 8×10⁶ cells/mL. In some aspects, the cell viability exhibits a mean peak viable cell density (VCD) of about 10×10⁶ cells/mL. In some aspects, the cell viability exhibits a mean peak viable cell density (VCD) of about 10.5×10⁶ cells/mL. In some aspects, the cell viability exhibits a mean peak viable cell density (VCD) of about 11×10⁶ cells/mL. In some aspects, the cell viability exhibits a mean peak viable cell density (VCD) of about 11.5×10⁶ cells/mL. In some aspects, the cell viability exhibits a mean peak viable cell density (VCD) of about 12×10⁶ cells/mL. In some aspects, the cell viability exhibits a mean peak viable cell density (VCD) of about 12.5×10⁶ cells/mL. In some aspects, the cell viability exhibits a mean peak viable cell density (VCD) of about 13×10⁶ cells/mL. In some aspects, the cell viability exhibits a mean peak viable cell density (VCD) of about 13.5×10⁶ cells/mL. In some aspects, the cell viability exhibits a mean peak viable cell density (VCD) of about 14×10⁶ cells/mL. In some aspects, the cell viability exhibits a mean peak viable cell density (VCD) of about 14.5×10⁶ cells/mL. In some aspects, the cell viability exhibits a mean peak viable cell density (VCD) of about 15×10⁶ cells/mL.

The methods of the present disclosure are also useful for controlling and/or monitoring the protein titer. The titer can be measured at one or more time points after a certain period of culturing cells in a bioreactor (to induce protein production in the bioreactor), i.e., during a protein production phase or protein induction phase. Titer can also be measured at the moment of culture harvest prior to downstream processing. In some aspects, the titer is measured during the protein induction phase after at least about a week from the start of the protein induction phase. In some aspects, the titer is measured during the protein induction phase after at least about 10 days, at least about two weeks, or at least about three weeks from the start of the protein induction phase. In some aspects, the titer is measured after a period of about 14 days from the start of the protein induction phase. In some aspects, the titer exhibits a mean day 14 titer between about 1.5 g/L to about 3.5 g/L. In some aspects, the titer exhibits a mean day 14 titer between about 1.5 g/L to about 3 g/L. In some aspects, the titer exhibits a mean day 14 titer between about 2 g/L to about 3 g/L. In some aspects, the titer exhibits a mean day 14 titer between about 2 g/L to about 2.5 g/L. In some aspects, the titer exhibits a mean day 14 titer between about 2.5 g/L to about 3 g/L. In some aspects, the titer exhibits a mean 14 day titer of about 2 g/L. In some aspects, the titer exhibits a mean 14 day titer of about 2.5 g/L. In some aspects, the titer exhibits a mean 14 day titer of about 3 g/L. In some aspects, the titer exhibits a mean 14 day titer of about 3.5 g/L.

In some aspects, the cells that are capable of expressing a protein, e.g., recombinant protein, are mammalian cells. In some aspects, the cells are eukaryotic cells. In some aspects, the cells are mammalian cells. In some aspects, the cells are selected from Chinese hamster ovary (CHO) cells, HEK293 cells, mouse myeloma (NS0), baby hamster kidney cells (BHK), monkey kidney fibroblast cells (COS-7), Madin-Darby bovine kidney cells (MDBK), and any combination thereof. In one aspect, the cells are Chinese hamster ovary cells. In some aspects, the cells are insect cells, e.g., Spodoptera frugiperda cells. In other aspects, the cells are mammalian cells. Such mammalian cells include but are not limited to CHO, VERO, BHK, Hela, MDCK, HEK 293, NIH 3T3, W138, BT483, Hs578T, HTB2, BT20 and T47D, NS0, CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In some aspects, the mammalian cells are CHO cells. In some aspects the CHO cell is CHO-DG44, CHOZN, CHO/dhfr-, CHOK1SV GS-KO, or CHO-S. In some aspects, the CHO cell is CHO-DG4. In some aspects, the CHO cell is CHOZN. Other suitable CHO cell lines disclosed herein include CHO-K (e.g., CHO K1), CHO pro3-, CHO P12, CHO-K1/SF, DUXB11, CHO DUKX; PA-DUKX; CHO pro5; DUK-BII or derivatives thereof.

### IIA. pH

In some aspects, the methods of the present disclosure involve improving or controlling protein production by modifying pH of the process. In some aspects, the methods involve modifying the pH set point of a protein production phase during a cell culture process. The regulation of intracellular pH is a fundamental physiological process of great significance to the growth and metabolism of cells. Since intracellular pH has wide-ranging consequences for the transport of nutrients and hormones, and for enzymatic reactions in the cells, cells devote a lot of energy to the regulation of cytoplasmic pH. Furthermore, pH plays a role in the glycosylation rates and profiles of protein produced by the cell. The present disclosure provides the surprising effect on the protein yield and/or on the glycosylation of the protein by controlling a pH set point in a protein production stage.

In some aspects, the pH set point useful for the present disclosure is between about 7.1 and about 7.2. In some aspects, the pH set point is about 7.15. In some aspects, the pH set point is 7.25. In some aspects, the pH set point is 7.15. In some aspects, the pH set point is 7.05. In some aspects, the pH set point is 7.0. In some aspects, the pH set point is 6. In some aspects, the pH set point is 6.1. In some aspects, the pH set point is 6.2. In some aspects, the pH set point is 6.3. In some aspects, the pH set point is 6.4. In some aspects, the pH set point is 6.5. In some aspects, the pH set point is 6.6. In some aspects, the pH set point is 6.7. In some aspects, the pH set point is 6.8. In some aspects, the pH set point is 6.9.

### IIB. Temperature

The temperature of a production vessel such as a bioreactor can be another important aspect of bioproduction because the temperature of the bioreactor plays a role in cell growth, viable cell density, cell longevity and/or glycosylation activity of glycosylating enzymes inside a cell. Temperature changes can significantly affect the rate of enzymatic reactions within the cell, denature proteins, and/or cause other effects on a cell culture. Cells can be cultured at an initial temperature set point such as 37° C., for example, to encourage maximum viable cell density, and then the temperature can be modified to another temperature set point (i.e., a second temperature set point or a final temperature set point) to prolong cell longevity or to enhance desired glycosylation activity within the cell. One or more temperature set points can be used during the various phases of an upstream production process to improve the overall cell density, protein yield, or protein glycosylation profile of a protein of interest. In some aspects, the methods are directed to one or more temperature adjustments during protein production. Temperature adjustments can be a decrease of operating temperature during a manufacturing process. Temperature adjustments can also be an increase of operating temperature during a manufacturing process. In some aspects, the methods of the present disclosure use at least one, at least two, at lease three, or at least four temperature adjustments during a manufacturing process.

The methods of the present disclosure are also related to controlling cell growth rate, cell viability, viable cell density and/or titer of cells for producing a protein. The initial temperature set point is important for creating reactor conditions conducive to cellular expansion and growth of the cells during the log growth phase. After the initial log phase, a second temperature set point that is lower than the initial set point is used to reduce the cellular expansion conditions to prevent overgrowth of the cell culture, which would lead to undesirable cell densities and a subsequent loss in total cell viability. In some aspects, the methods of the present disclosure involve culturing the cells in a bioreactor for an induction phase under an initial temperature set point of about 36° C., subsequently culturing the cells in a second temperature set point of 33° C., and finally culturing the cells at a final temperature set point of 31° C. In some aspects, the methods of the present disclosure use at least two, at least three, at least four, or at least five temperature set points, e.g., an initial temperature set point, a final set point, or more set points after the initial temperature set point but before the final set point. In some aspects, the methods of the present disclosure use at least three temperature set points, i.e., an initial temperature set point, a second temperature set point, and a final temperature set point.

In some aspects, the initial temperature set point for the present method is about 37° C. and a second temperature set point is lower than about 36° C. In some aspects, the initial temperature set point is about 36° C. and a second temperature set point is lower than the first temperature set point, e.g., about 35° C., about 34° C., about 33° C., about 32° C., or about 31° C. In some aspects, the initial temperature set point is about 37° C. and a second temperature set point is lower than about 34° C. In some aspects, the initial temperature set point is about 36° C. and a second temperature set point is lower than about 35° C., about 34° C., or about 33° C. In some aspects, the initial temperature set point is lower than about 36.5° C. and the final temperature set point is about 31° C. In some aspects, the initial temperature set point is about 36.0° C. and the final temperature set point is about 31° C. In some aspects, the initial temperature set point is lower than about 35.5° C. and the final temperature set point is about 31° C. In some aspects, the initial temperature set point is lower than about 35.0° C. and the final temperature set point is about 31° C. In some aspects, the initial temperature set point is lower than about 36.5° C., a second temperature set point is about 33° C., and a final temperature set point is lower than about 33° C. or about 32° C. In some aspects, the initial temperature set point is about 36.0° C., a second temperature set point is about 33° C., and a final temperature set point is lower than about 33° C. or about 32° C. In some aspects, the initial temperature set point is about 36.0° C., a second temperature set point is about 33° C., and a final temperature set point is lower than about 32° C. In some aspects, the initial temperature set point is about 36.0° C., a second temperature set point is about 33° C., and a final temperature set point is about 31° C.

The methods of the present disclosure can comprise an initial temperature set point, a second temperature set point, and a third and/or final set point. The initial, second, and third and/or final temperature set points are used to further control steady-state cell density during a manufacturing process, control viable cell percentages, manage cell-cycle divisions of the culture, and/or alter the glycosylation rates and glycosylation profile of produced proteins. In some aspects, the third temperature set point is lower than the second temperature set point. In some aspects, the initial temperature set point is a temperature between 34° C. and 37° C., e.g., 34° C., 35° C., 36° C., or 37° C.; the second temperature set point is a temperature between 32° C. and 34° C., e.g., 32° C., 33° C., or 34° C.; and the final temperature set point is a temperature between 30° C. and 32° C., e.g., 30° C., 31° C., or 32° C., wherein the second temperature set point is lower than the first temperature set point and the final temperature set point is lower than the second temperature set point. In some aspects, the initial temperature set point is a temperature between 34° C. and 37° C., e.g., 34° C., 35° C., 36° C., or 37° C.; the second temperature set point is a temperature between 32° C. and 34° C., e.g., 32° C., 33° C., or 34° C.; and the final temperature set point is a temperature between 30° C. and 32° C., e.g., 30° C., 31° C., or 32° C., wherein the first temperature set point is not 37° C., the second temperature set point is not 34° C., and/or the final temperature set point is not 32° C.

The methods of the present disclosure can comprise an initial temperature set point, a second temperature set point, a third temperature set point, and optionally a fourth temperature set point, optionally a fifth temperature set point, and optionally a sixth temperature set point. These fourth, fifth, and sixth temperature set points are used to further control steady-state cell density during a manufacturing process, control viable cell percentages, manage cell-cycle divisions of the culture, and/or alter the glycosylation rates and glycosylation profile of produced proteins. In some aspects, the methods further comprise setting an optional fourth temperature set point, an optional fifth temperature set point, an optional sixth temperature set point, wherein the optional fourth temperature set point, the fifth temperature set point, and/or the sixth temperature set point are lower than the third temperature set point. In some aspects, the methods further comprises setting an optional fourth temperature set point, an optional fifth temperature set point, and an optional sixth temperature set point, wherein the optional fourth temperature set point, the fifth temperature set point, and/or the sixth temperature set point are higher than the third temperature set point. In some aspects, the fourth, fifth, or sixth temperature set point, the fifth temperature set point, and/or the sixth temperature set point is about 30° C., about 31° C., about 32° C., about 33° C., about 34° C., about 35° C., about 36° C., about 37° C., about 38° C., about 39° C., or about 40° C. In some aspects, the fourth, fifth, or sixth temperature set point, the fifth temperature set point, and/or the sixth temperature set point is about 30° C. In some aspects, the fourth, fifth, or sixth temperature set point is about 31° C. In some aspects, the fourth, fifth, or sixth temperature set point is about 32° C. In some aspects, the fourth, fifth, or sixth temperature set point is about 33° C. In some aspects, the fourth, fifth, or sixth temperature set point is about 34° C. In some aspects, the fourth, fifth, or sixth temperature set point is about 35° C. In some aspects, the fourth, fifth, or sixth temperature set point is about 36° C. In some aspects, the fourth, fifth, or sixth temperature set point is about 37° C. In some aspects, the fourth, fifth, or sixth temperature set point is about 38° C. In some aspects, the fourth, fifth, or sixth temperature set point is about 39° C. In some aspects, the fourth, fifth, or sixth temperature set point is about 40° C.

Apart from the temperature set points, the methods of the present disclosure also involve modification of the timing of the temperature set points to carry out a temperature shift within a specific time window. The timing of the temperature shift is important to control the cell density, cell growth, and protein production characteristics of the cell culture during the upstream process. In some aspects, the methods of the present disclosure are related to a method of improving the yield of a protein in cells, comprising culturing the cells in a bioreactor under suitable conditions, wherein the suitable conditions comprise (i) an initial temperature set point of 36.0° C. and a second temperature set point lower than about 36° C.; (ii) an initial temperature set point lower than about 36.5° C. and a final temperature set point of about 31° C.; or (iii) an initial temperature set point lower than about 36.5° C., a second temperature set point of about 33° C., and a final temperature set point lower than about 33° C. In some aspects, the methods of the present disclosure are related to a method of improving the yield of a protein in cells, comprising culturing the cells in a bioreactor under suitable conditions, wherein the suitable conditions comprise (i) an initial temperature set point of 36.0° C. and a second temperature set point lower than 34° C.; (ii) an initial temperature set point lower than 36.5° C. and a final temperature set point of 31° C.; or (iii) an initial temperature set point lower than 36.5° C., a second temperature set point of 32° C., and a final temperature set point lower than 32° C.

In some aspects, the cells are cultured in a bioreactor under suitable conditions, comprising (i) an initial temperature set point higher than about 35° C. but lower than about 37° C. and a second temperature set point higher than about 32° C. but lower than about 34° C., (ii) an initial temperature set point higher than about 35.5° C. but lower than about 37.5° C. and a second temperature set point higher than about 32° C. but lower than about 34° C., and (iii) an initial temperature set point higher than about 35° C. but lower than about 37° C., a second temperature set point higher than about 32° C. but lower than about 34° C., and a final temperature set point higher than about 30° C. but lower than about 32° C.

The methods of the present disclosure are also useful for improving yield of a protein in cells by adjusting the temperature set point in the bioreactor after an initial temperature set point. In some aspects, the initial temperature set point is higher than about 35° C. and lower than 37° C., e.g., about 36° C., the second temperature set point is higher than about 32° C. and lower than about 34° C., about 33° C., and the third temperature set point is higher than about 30° C. and lower than about 32° C., e.g., about 31° C., wherein the second temperature set point occurs between about 120 hours to about 168 hours, e.g., about 5 days, about 6 days, or about 7 days, after the initial temperature set point. In some aspects, the initial temperature set point is higher than about 35° C. and lower than 37° C., e.g., about 36° C., the second temperature set point is higher than about 32° C. and lower than about 34° C., about 33° C., and the third temperature set point is higher than about 30° C. and lower than about 32° C., e.g., about 31° C., wherein the second temperature set point occurs between about 84 hours, at about 90 hours, at about 96 hours, at about 102 hours, at about 108 hours, about 114 hours, about 120 hours, about 126 hours, about 132 hours, about 138 hours, about 144 hours, about 150 hours, about 156 hours, about 162 hours, or about 168 hours after the initial temperature set point. In some aspects, the second temperature set point occurs at from about 72 hours to about 168 hours. In some aspects, the second temperature set point occurs at from about 96 hours to about 168 hours. In some aspects, the second temperature set point occurs at from about 96 hours to about 144 hours.

In some aspects, the initial temperature set point is higher than about 35° C. and lower than 37° C., e.g., about 36° C., the second temperature set point is higher than about 32° C. and lower than about 34° C., about 33° C., and the third temperature set point is higher than about 30° C. and lower than about 32° C., e.g., about 31° C., wherein the second temperature set point occurs at about 120 hours, 5 days, after the initial temperature set point. In some aspects, the initial temperature set point is higher than about 35° C. and lower than 37° C., e.g., about 36° C., the second temperature set point is higher than about 32° C. and lower than about 34° C., about 33° C., and the third temperature set point is higher than about 30° C. and lower than about 32° C., e.g., about 31° C., wherein the second temperature set point occurs at about 144 hours, 6 days, after the initial temperature set point. In some aspects, the initial temperature set point is higher than about 35° C. and lower than 37° C., e.g., about 36° C., the second temperature set point is higher than about 32° C. and lower than about 34° C., about 33° C., and the third temperature set point is higher than about 30° C. and lower than about 32° C., e.g., about 31° C., wherein the second temperature set point occurs at about 168 hours, 7 days, after the initial temperature set point. In some aspects, the second temperature set point occurs at about 192 hours, e.g., 8 days, after the initial temperature set point.

The methods of the present disclosure are also used to control the transition timing to the final temperature set point. In some aspects, the initial temperature set point is higher than about 35° C. and lower than 37° C., e.g., about 36° C., the second temperature set point is higher than about 32° C. and lower than about 34° C., about 33° C., and the final temperature set point is higher than about 30° C. and lower than about 32° C., e.g., about 31° C., wherein the final temperature set point occurs at from about 168 hours to about 312 hours, about 7 days, about 8 days, about 9 days, about 10 days, about 11 days, about 12 days, or about 13 days, after the initial temperature set point, from 7 days to 12 days, from 8 days to 13 days, from 9 days to 13 days, from 8 days to 12 days, from 8 days to 11 days. In some aspects, the initial temperature set point is higher than about 35° C. and lower than 37° C., e.g., about 36° C., the second temperature set point is higher than about 32° C. and lower than about 34° C., about 33° C., and the final temperature set point is higher than about 30° C. and lower than about 32° C., e.g., about 31° C., wherein the final temperature set point occurs at from about 192 hours to about 300 hours. In some aspects, the final temperature set point occurs at from about 216 hours to about 288 hours. In some aspects, the final temperature set point occurs at from about 216 hours to about 264 hours.

### IIC. Viable Cell Density

The initial viable cell density (VCD) or seed density of cells at the outset of a bioreactor process can be an important aspect of the upstream growth process as the initial viable cell density (VCD) impacts the steady-state and/or maximum viable cell density of the cell culture during production. A higher initial cell density can lead to a much higher number of cells produced during the initial growth phase of the upstream process, and can ultimately lead to faster cellular death and a shorter bioreactor run time. The bioreactor production process can be shortened, but in some cases, the post-translational modifications made to proteins produced by the process can be affected, as a shorter bioreactor run time can lead to altered residence time of the proteins in the Golgi, and therefore altered post-translational modifications, such as changes to the glycosylation pattern of the protein. Because of the wide implications that the initial cell density can have on the overall yield and quality output of the protein, the initial viable cell density is an important parameter for upstream protein production.

In some aspects, the methods of the present disclosure involve seeding a bioreactor with an initial viable cell density (VCD). In some aspects, the initial viable cell density (VCD) set point is between about 0.45×10⁶ cells/mL and about 1.35×10⁶ cells/mL. In some aspects, the initial viable cell density (VCD) set point is between about 0.45×10⁶ cells/mL and about 1.3×10⁶ cells/mL. In some aspects, the initial viable cell density (VCD) set point is between about 0.45×10⁶ cells/mL and about 1.25×10⁶ cells/mL. In some aspects, the initial viable cell density (VCD) set point is between about 0.45×10⁶ cells/mL and about 1.2×10⁶ cells/mL. In some aspects, the initial viable cell density (VCD) set point is between about 0.45×10⁶ cells/mL and about 1.15×10⁶ cells/mL. In some aspects, the initial viable cell density (VCD) set point is between about 0.45×10⁶ cells/mL and about 1.1×10⁶ cells/mL. In some aspects, the initial viable cell density (VCD) set point is between about 0.45×10⁶ cells/mL and about 1.05×10⁶ cells/mL. In some aspects, the initial viable cell density (VCD) set point is between about 0.5×10⁶ cells/mL and about 1.0×10⁶ cells/mL. In some aspects, the initial viable cell density (VCD) set point is between about 0.55×10⁶ cells/mL and about 1.0×10⁶ cells/mL. In some aspects, the initial viable cell density (VCD) set point is between about 0.50×10⁶ cells/mL and about 0.90×10⁶ cells/mL. In some aspects, the initial viable cell density (VCD) set point is between about 0.55×10⁶ cells/mL and about 0.90×10⁶ cells/mL. In some aspects, the initial viable cell density (VCD) set point is between about 0.55×10⁶ cells/mL and about 0.85×10⁶ cells/mL. In some aspects, the initial viable cell density (VCD) set point is between about 0.55×10⁶ cells/mL and about 0.8×10⁶ cells/mL. In some aspects, the initial viable cell density (VCD) set point is between about 0.6×10⁶ cells/mL and about 1.0×10⁶ cells/mL. In some aspects, the initial viable cell density (VCD) set point is between about 0.6×10⁶ cells/mL and about 0.75×10⁶ cells/mL. In some aspects, the initial viable cell density (VCD) set point is between about 0.65×10⁶ cells/mL and about 0.75×10⁶ cells/mL.

### IID. Cell Feed Time

The methods of the present disclosure can also be achieved through altering a cell feed time to affect or control growth conditions of the cells. The timing of a feeding process is important to produce the desired growth properties of a cell culture production process, such as cell density. The optimal feeding strategy in bioreactors depends on the structure of the reaction kinetics and the interaction between the different reactions, such as protein synthesis and post-translational modification of those proteins (i.e., glycosylation). Both overfeeding and underfeeding a cellular population are detrimental to cell growth and product formation, and therefore the timing of the feeds can be important to ensure maximum product yield. Underfeeding of cultures can lead to nutrient depletion and cell death, while overfeeding can lead to an excess of nutrients, an increase in osmolality and cellular stress in dense cellular environments, and undesirable post-translational modifications of a protein of interest.

In some aspects, the cell feed time is from about 24 hours to about 100 hours after induction. In some aspects, the cell feed time is from about 48 hours to about 100 hours after induction. In some aspects, the cell feed time is from about 48 hours to about 72 hours after induction. In some aspects, the cell feed time is from about 48 hours to about 96 hours after induction. In some aspects, the cell feed time is from about 72 hours to about 96 hours after induction. In some aspects, the cell feed time is from about 66 hours to about 84 hours after induction. In some aspects, the cell feed time is about 24 hours after induction. In some aspects, the cell feed time is about 48 hours after induction. In some aspects, the cell feed time is about 66 hours after induction. In some aspects, the cell feed time is about 72 hours after induction. In some aspects, the cell feed time is about 84 hours after induction. In some aspects, the cell feed time is about 96 hours after induction.

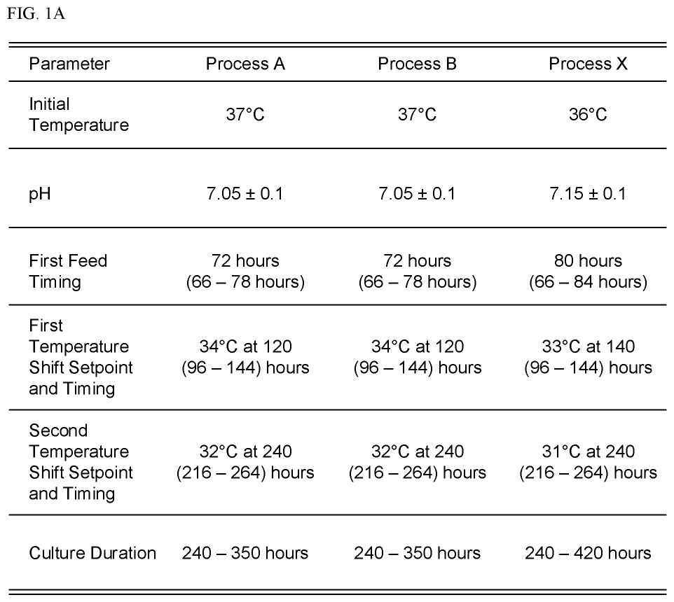

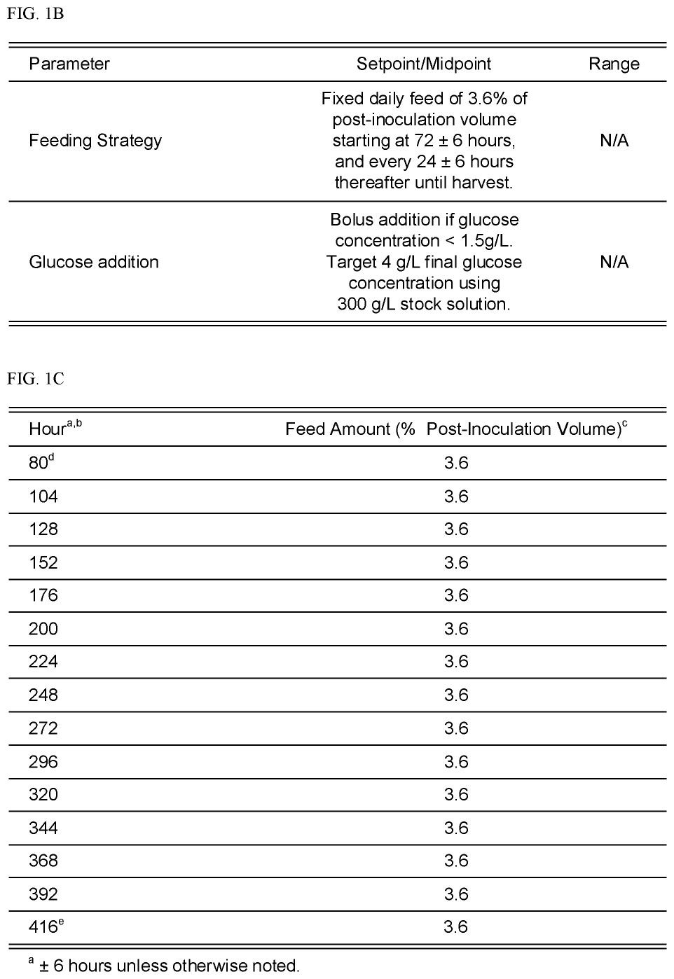

In some aspects, a first cell feed time is about 80 hours after induction and one or more subsequent feed times occur about 24 hours thereafter. In some aspects, the subsequent feed times are 104 hours after induction, 128 hours after induction, 152 hours after induction, 176 hours after induction, 200 hours after induction, 224 hours after induction, 248 hours after induction, 272 hours after induction, 296 hours after induction, 320 hours after induction, 344 hours after induction, 368 hours after induction, 392 hours after induction, and/or 416 hours after induction. In some aspects, a first cell feed time is about 74 hours to about 86 hours after induction and one or more subsequent feed times occur about 24 hours thereafter. In some aspects, the subsequent feed times are about 98 hours to about 110 hours after induction. In some aspects, the subsequent feed times are about 122 hours to about 134 hours after induction. In some aspects, the subsequent feed times are about 146 hours to about 158 hours after induction. In some aspects, the subsequent feed times are about 170 hours to about 182 hours after induction. In some aspects, the subsequent feed times are about 194 hours to about 206 hours after induction. In some aspects, the subsequent feed times are about 218 hours to about 230 hours after induction. In some aspects, the subsequent feed times are about 242 hours to about 254 hours after induction. In some aspects, the subsequent feed times are about 266 hours to about 278 hours after induction. In some aspects, the subsequent feed times are about 290 hours to about 302 hours after induction. In some aspects, the subsequent feed times are about 314 hours to about 326 hours after induction. In some aspects, the subsequent feed times are about 338 hours to about 350 hours after induction. In some aspects, the subsequent feed times are about 362 hours to about 374 hours after induction. In some aspects, the subsequent feed times are about 386 hours to about 396 hours after induction. In some aspects, the subsequent feed times are about 410 hours to about 422 hours after induction. In some aspects, the subsequent feed times are according to FIG. 1B.

### IIE. Combination of Conditions

In some aspects, the methods of the present disclosure comprise any combination of the above listed conditions. In some aspects, the methods comprise two or more conditions selected from the group consisting of: (i) an initial temperature set point between about 35° C. and about 37° C., e.g., about 36° C., a second temperature set point between 32° C. and about 34° C., e.g., about 33° C., and a third temperature set point between about 30° C. and about 32° C., e.g., about 31° C.; (ii) a pH set point of 7.15; and (iii) an initial viable cell density (VCD) set point between about 0.65×10⁶ cells/mL and about 0.75×10⁶ cells/mL.

In some aspects, the methods comprise (i) an initial temperature set point between about 35° C. and about 37° C., e.g., about 36° C., a second temperature set point between 32° C. and about 34° C., e.g., about 33° C., and a third temperature set point between about 30° C. and about 32° C., e.g., about 31° C. and (ii) a pH set point of 7.15.

In some aspects, the methods comprise (i) an initial temperature set point of about 36° C., a second temperature set point of about 33° C., and a third temperature set point of about 31° C. and (ii) an initial viable cell density (VCD) set point between about 0.65×10⁶ cells to about 0.75×10⁶ cells (e.g., 0.70×10⁶ cells).

In some aspects, the methods comprise (i) an initial temperature set point between about 35° C. and about 37° C., e.g., about 36° C., a second temperature set point between 32° C. and about 34° C., e.g., about 33° C., and a third temperature set point between about 30° C. and about 32° C., e.g., about 31° C., (ii) a pH set point of 7.15, and (iii) an initial viable cell density (VCD) set point between about 0.65×10⁶ cells to about 0.75×10⁶ cells (e.g., 0.70×10⁶ cells).

In some aspects, the methods comprise (i) an initial temperature set point between about 35° C. and about 37° C., e.g., about 36° C., a second temperature set point between 32° C. and about 34° C., e.g., about 33° C., and a third temperature set point between about 30° C. and about 32° C., e.g., about 31° C., (ii) a pH set point of 7.15; (iii) an initial viable cell density (VCD) set point between about 0.65×10⁶ cells to about 0.75×10⁶ cells (e.g., 0.70×10⁶ cells); and (iv) a first feed time between about 66 to about 84 hours.

In some aspects, the methods comprise (i) an initial temperature set point between about 35° C. and about 37° C., e.g., about 36° C., a second temperature set point between 32° C. and about 34° C., e.g., about 33° C., and a third temperature set point between about 30° C. and about 32° C., e.g., about 31° C., (ii) a pH set point of 7.15; (iii) an initial viable cell density (VCD) set point between about 0.65×10⁶ cells to about 0.75×10⁶ cells (e.g., 0.70×10⁶ cells); and (iv) a first feed time of about 80 hours.

In some aspects, the methods comprise (i) an initial temperature set point between about 35° C. and about 37° C., e.g., about 36° C., a second temperature set point between 32° C. and about 34° C., e.g., about 33° C., and a third temperature set point between about 30° C. and about 32° C., e.g., about 31° C., (ii) a pH set point of 7.15; (iii) an initial viable cell density (VCD) set point between about 0.65×10⁶ cells to about 0.75×10⁶ cells (e.g., 0.70×10⁶ cells); and (iv) a first feed time between about 66 to about 84 hours.

In some aspects, the methods comprise (i) an initial temperature set point between about 35° C. and about 37° C., e.g., about 36° C., a second temperature set point between 32° C. and about 34° C., e.g., about 33° C., and a third temperature set point between about 30° C. and about 32° C., e.g., about 31° C., (ii) a pH set point of 7.15; (iii) an initial viable cell density (VCD) set point between about 0.65×10⁶ cells to about 0.75×10⁶ cells (e.g., 0.70×10⁶ cells); and (iv) a first feed time of about 80 hours.

### IIF. Culture Medium

The cells producing a protein of interest can be grown in a cell culture medium. The term “culture media” (used interchangeably with “culture medium”) as use herein refers to a nutritive composition that aids in sustaining, propagating, and/or differentiating cells. The term “culture media” refers to any medium which is capable of supporting growth, maintenance, propagation, or expansion of cells in an artificial in vitro environment outside of a multicellular organism or tissue. Cell culture medium can be optimized for a specific cell culture use, including, for example, cell culture growth medium which is formulated to promote cellular growth, or cell culture production medium which is formulated to promote recombinant protein production. The culture medium supplies standard inorganic salts, such as zinc, iron, magnesium, calcium and potassium, as well as trace elements, vitamins, an energy source, a buffer system, and essential amino acids. Exemplary culture media include, but are not limited to, Iscove's Modified Dulbecco's Medium, RPMI 1640, Minimal Essential Medium-alpha (MEM-alpha), Dulbecco's Modification of Eagle's Medium (DMEM), DME/F12, alpha MEM, Basal Medium Eagle with Earle's BSS, DMEM high Glucose with L-Glutamine, DMEM high glucose without L-Glutamine, DMEM low Glucose without L-Glutamine, DMEM:F12 1:1 with L-Glutamine, GMEM (Glasgow's MEM), GMEM with L-glutamine, Grace's Complete Insect Medium, Grace's Insect Medium without FBS, F-10, F-12, Ham's F-10 with L-Glutamine, Ham's F-12 with L-Glutamine, IMDM with HEPES and L-Glutamine, IMDM with HEPES and without L-Glutamine, IPL-41 Insect Medium, L-15 (Leibovitz)(2×) without L-Glutamine or Phenol Red, L-15 (Leibovitz) without L-Glutamine, McCoy's 5A Modified Medium, Medium 199, MEM Eagle without L-Glutamine or Phenol Red (2×), MEM Eagle-Earle's BSS with L-glutamine, MEM Eagle-Earle's BSS without L-Glutamine, MEM Eagle-Hanks BSS without L-Glutamine, NCTC-109 with L-Glutamine, Richter's CM Medium with L-Glutamine, RPMI 1640 with HEPES, L-Glutamine and/or Penicillin-Streptomycin, RPMI 1640 with L-Glutamine, RPMI 1640 without L-Glutamine, Schneider's Insect Medium, or any other media suitable for the present method. Additionally, culture media as described herein include, but are not limited to, chemically defined media, hydrolysate-containing media, and simple media.

The methods of the present disclosure can be performed in a variety of vessel types to accommodate various protein production strategies. The methods of the present disclosure can involve a fed-batch culture. Fed-batch culture is a method of culturing cells in which additional components are provided to the culture at some time subsequent to the beginning of the culture process. A fed-batch culture can be started using a basal medium. The culture medium with which additional components are provided to the culture at some time subsequent to the beginning of the culture process is a feed medium. A fed-batch culture is typically stopped at some point and the cells and/or components in the medium are harvested and optionally purified. The methods of the present disclosure can involve a perfusion culture. Perfusion culture involves a continuous flow of a physiological nutrient solution at a steady rate, through or over a population of cells. As perfusion systems generally involve the retention of the cells within the culture unit, perfusion cultures characteristically have relatively high cell densities, but the culture conditions are difficult to maintain and control. In addition, since the cells are grown to and then retained within the culture unit at high densities, the growth rate typically continuously decreases over time, leading to the late exponential or even stationary phase of cell growth. In some aspects, the methods of the present disclosure involve a batch culture process. In some aspects, the methods of the present disclosure involve a batch-fed culture process. In some aspects, the methods of the present disclosure involve a perfusion culture process.

In some aspects, the culture media suitable for the present methods can be supplemented with a feed medium. In some aspects, the feed medium is a chemically defined feed medium. In some aspects, chemically defined feed media (or CDFM) or medium refers to media which contain one or more nutrients whose chemical composition and relative concentrations are known, and which is added to the culture media beginning at some time after inoculation. CDFM is sometimes used interchangeably with “concentrated feed media,” “enriched media,” “highly concentrated feed media” or “super concentrated feed media.” CDFM is supplied to the culturing vessel continuously or in discrete increments, to the culture media during culturing, with or without periodic cell and/or product harvest before termination of culture. CDFM can be individually formulated comprise a unique blend of amino acids, vitamins, trace minerals, and organic compounds, at enriched amounts to serve as a feed media to cell culture media. Alternatively, commercially available CDFM can be used. Some examples of commercially available CDFM include, but are not limited to, IS CHO Feed-CD (Irvine Scientific), BALANCD™ CHO Feed Medium (1-3) (Irvine Scientific), IS-CHO-V™ (Irvine Scientific), IS-CHO-CD XP™ with Hydrolysate Blend (Irvine Scientific), CHO Feed Bioreactor Supplement (Sigma-Aldrich), CHO CD EFFICIENT FEED™ B nutrient supplement (Life Technologies).

In some aspects, the cells used in the present invention are prokaryote, yeast, or higher eukaryote cells. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One suitable E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In certain embodiments, the cells are eukaryotic microbes such as filamentous fungi or yeast. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

In some aspects, the cells are derived from multicellular organisms. In particular embodiments, the cells are invertebrate cells from plant and insect cells. Non-limiting examples include cells derived from Spodoptera frugiperda (caterpillar), Aedes aegypti (mosquito), Aedes albopictus (mosquito), Drosophila melanogaster (fruitfly), Bombyx mori, cotton, corn, potato, soybean, petunia, tomato, and tobacco can also be utilized.

In some aspects, the cells are mammalian cells. For example, the cells are Chinese Hamster Ovary (CHO cells) (including dhfr-CHO cells, described in Urlaub and Chasin, (1980) PNAS USA 77:4216-4220, used with a DHFR selectable marker, e.g., as described in Kaufman and Sharp (1982) Mol. Biol. 159:601-621, the entire teachings of which are incorporated herein by reference), NSO myeloma cells, COS cells and SP2 cells. Other non-limiting examples of mammalian cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), the entire teachings of which are incorporated herein by reference.

In some aspects, the cells are transformed with expression or cloning vectors for producing products or portions thereof and cultured as appropriate for inducing promoters, selecting transformants, or amplifying the genes encoding the desired sequences. In some aspects, standard molecular biology techniques are used to prepare the recombinant expression vector, transfect the cells, select for transformants, culture the cells and recover the product from the culture medium. In some aspects, the cell culture media described herein can be used as culture media for hybridoma cells, monoclonal antibody producing cells, virus-producing cells, transfected cells, cancer cells and/or recombinant peptide producing cells.

The cells of the present disclosure can be cultured under suitable conditions for suitable periods of time, conditions that depend on the type(s) of cells being cultured and the product being produced. In some aspects, the cells are cultured for about two to about fourteen days. In some aspects, the cells are cultured from about four to about ten days.

In some aspects, the cell culture is suspension culture or adherent culture. The term “suspension culture” refers to cells in culture in which the majority or all of the cells in culture are present in suspension, and the minority or none of the cells in the culture vessel are attached to the vessel surface or to another surface within the vessel (adherent cells). The “suspension culture” can have greater than about 50%, 60%, 65%, 75%, 85%, or 95% of the cells in suspension, not attached to a surface on or in the culture vessel. The term “adherent culture” refers to cells in culture in which the majority or all of the cells in culture are present attached to the vessel surface or to another surface within the vessel, and the minority or none of the cells in the culture vessel are in suspension. The “adherent culture” can have greater than 50%, 60%, 65%, 75%, 85%, or 95% of the cells adherent.

The culture medium for producing recombinant proteins can include one or more metal ions suitable for cell culture media. In some aspects, the metal ions that can be included in the cell culture are, without limitation, Ag⁺, Au³⁺, Cd²⁺, Cu²⁺, Ga³⁺, I³⁺, Ni²⁺, Pd²⁺, Zn²⁺, and Mn²⁺. In some aspects, the concentration of certain metal ions, e.g., manganese, at the outset of a bioreactor process can impact the growth and/or steady-state cell culture kinetics during production. Furthermore, metal ions, e.g., manganese, can affect the throughput of various enzymes involved in glycosylation of protein produced in a bioreactor. Among others, Manganese is a known co-factor for several glycotransferases in mammalian cells, including N-acetyl-glucosaminyl-transferase and β-1,4-galactosyl-transferase. Thus, not being bound by any theory, the adjustment of manganese concentration in a bioreactor process can affect the glycosylation distribution and glycosylation profile of protein. In some aspects, manganese is supplemented to a bioreactor, i.e., a fed batch bioreactor. In some aspects, manganese is supplemented to a bioreactor at the start of or during a protein induction phase.

In some aspects, the manganese for the present methods is at a concentration of at least about 30 mg/mL, at least about 50 mg/mL, at least about 80 mg/mL, at least about 100 mg/mL, at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL, at least about 200 mg/mL, at least about 220 mg/mL, at least about 240 mg/mL, at least about 260 mg/mL, at least about 280 mg/mL, or at least about 300 mg/mL. In some aspects, the methods of the present disclosure involve adjusting the manganese concentration to about 30-300 mg/mL. In some aspects, the manganese concentration is from about 30 mg/mL to about 280 mg/mL. In some aspects, the manganese concentration is from about 50 mg/mL to about 280 mg/mL. In some aspects, the manganese concentration is from about 50 mg/mL to about 260 mg/mL. In some aspects, the manganese concentration is from about 70 mg/mL to about 260 mg/mL. In some aspects, the manganese concentration is from about 90 mg/mL to about 240 mg/mL. In some aspects, the manganese concentration is from about 120 mg/mL to about 240 mg/mL. In some aspects, the manganese concentration is from about 120 mg/mL to about 220 mg/mL. In some aspects, the manganese concentration is from about 140 mg/mL to about 220 mg/mL. In some aspects, the manganese concentration is from about 140 mg/mL to about 200 mg/mL. In some aspects, the manganese concentration is from about 160 mg/mL to about 200 mg/mL. In some aspects, the manganese concentration is from about 160 mg/mL to about 180 mg/mL. In some aspects, the manganese concentration is about 160 mg/mL, about 160 mg/mL, or about 180 mg/mL.

Manganese concentrations can also be measured during an upstream cell culture process. Manganese can also be measured during a protein production phase or protein induction phase, i.e., directly in a bioreactor. In some aspects, the manganese is present at a concentration of about 1-20 parts per billion (ppb). In some aspects, the manganese is present at a concentration of about 1.6 ppb to about 15 ppb. In some aspects, the manganese is present at a concentration of about 2 ppb to about 10 ppb. In some aspects, the manganese is present at a concentration of about 2 ppb to about 6 ppb. In some aspects, the manganese is present at a concentration of about 2 ppb to about 4 ppb. In some aspects, the manganese is present at a concentration of about 1 ppb to about 3 ppb. In some aspects, the manganese is present at a concentration of about 1 ppb to about 2 ppb. In some aspects, the manganese is present at a concentration of about 3 ppb to about 10 ppb. In some aspects, the manganese is present at a concentration about 3 ppb to about 6 ppb. In some aspects, the manganese is present at a concentration of about 1.3 ppb. In some aspects, the manganese is present at a concentration of about 1.6 ppb. In some aspects, the manganese is present at a concentration of about 2 ppb. In some aspects, the manganese is present at a concentration of about 3 ppb. In some aspects, the manganese is present at a concentration of about 4 ppb. In some aspects, the manganese is present at a concentration of about 5 ppb. In some aspects, the manganese is present at a concentration of about 6 ppb.

The present disclosure also comprises a protein produced by the present methods. In some aspects, the protein produced by the present methods is a CTLA4-Fc fusion protein. In some aspects, the protein comprises belatacept. In some aspects, the protein produced by the present methods are glycosylated. In some aspects, the glycosylated proteins are shown elsewhere herein.

In some aspects, the present disclosure comprises cells that are cultured by the present methods. Also provided is a bioreactor that is useful for the present method. In some aspects, the bioreactor is maintained at: (a) an initial temperature set point of about 36° C., a second temperature set point of about 33° C., and a third temperature set point of about 31° C.; (b) a pH set point of about 7.15; and (c) an initial viable cell density (VCD) set point of about 0.70×10⁶ cells/mL.

### III. Glycosylation Profile

The methods and conditions of the present disclosure are useful for affecting, maintaining, controlling, and/or modifying the glycosylation profile of a protein produced by the present methods. In some aspects, the method controls a glycosylation profile of the protein. In some aspects, the glycosylation profile of the protein comprises one or more N-linked glycans.

The methods of the present disclosure can be achieved or substantiated via glycan analysis using a variety of methods, including a glycan release assay. The first step in glycan analysis of glyco-conjugates such as glycoproteins is the release of the sugars from the molecules to which they are attached. N-linked glycans on a glycoprotein can be released by an amidase such as Peptide-N-glycosidase F (PNGase F). Most methods for the analysis of oligosaccharides from biological sources require a glycan derivatization step: glycans can be derivatized to introduce a chromophore or fluorophore, facilitating detection after chromatographic or electrophoretic separation. Derivatization can also be applied to link charged or hydrophobic groups at the reducing end to enhance glycan separation and mass-spectrometric detection. Moreover, derivatization steps such as permethylation aim at stabilizing sialic acid residues, enhancing mass-spectrometric sensitivity, and supporting detailed structural characterization by (tandem) mass spectrometry.

In the specific case of belatacept, after glycan derivitization, an immunoglobulin-degrading enzyme, such as those from Streptococcus pyogenes (IdeS), can be used to digest the hinge region of IgG at one specific site just below the hinge region, resulting in a CTLA4 fragment and two Fc fragments. Glycan release, derivatization, and glycan analysis can then be performed on the separated fragments, wherein the Asn76 and Asn108 sites can be analyzed together (CTLA4 region), and the Asn207 (Fc region) site can be analyzed separately.

Mass spectrometry (“MS” or “mass-spec”) is an analytical technique used to measure the mass-to-charge ratio ions. This is achieved by ionizing the sample and separating ions of differing masses and recording their relative abundance by measuring intensities of ion flux. A typical mass spectrometer comprises three parts: an ion source, a mass analyzer, and a detector system. The ion source is the part of the mass spectrometer that ionizes the substance under analysis (the analyte). The ions are then transported by magnetic or electric fields to the mass analyzer that separates the ions according to their mass-to-charge ratio (m/z). Many mass spectrometers use two or more mass analyzers for tandem mass spectrometry (MS/MS). The detector records the charge induced or current produced when an ion passes by or hits a surface. A mass spectrum is the result of measuring the signal produced in the detector when scanning m/z ions with a mass analyzer.

A variety of N-linked glycans can be present in the glycosylation profile of a protein. In some aspects, the N-linked glycans comprise G0F, G1F, G2F, S1G1F, S1G2F, S2G2F, S1G3F, and/or S2G4F. Representative diagrams of G0F, G1F, G2F, S1G1F, S2G2F, S1G3F, and S2G4F can be seen in FIGS. 6A-6B. In some aspects, the methods of the present disclosure involve measuring the glycosylation profile during a protein induction phase after day 1, day 2, day 3, day 4, day 5, day 6, day 7, day 8, day 9, day 10, day 11, day 12, day 13, day 14, day 15, day 16, day 17, day 18, day 9, day 20, or day 21 of the start of the protein induction phase. In some aspects, the methods of the present disclosure involve measuring the glycosylation profile after day 7 of the start of the protein induction phase. In some aspects, the methods of the present disclosure involve measuring the glycosylation profile after day 14 of the start of the protein induction phase. In some aspects, the methods of the present disclosure involve measuring the glycosylation profile after day 21 of the start of the protein induction phase. In some aspects, the methods of the present disclosure involve measuring the glycosylation profile at cell culture harvest.

In some aspects, a glycosylated protein produced by the present methods is a CTLA4 protein. A CTLA4 molecule or CTLA4 extracellular domain can be fused to an Fc, wherein the molecule is referred to as CTLA4-Fc or CTLA4-Ig. An “Fc region” (fragment crystallizable region), “Fc domain,” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. The IgG isotype is divided in subclasses in certain species: IgG1, IgG2, IgG3 and IgG4 in humans, and IgG1, IgG2a, IgG2b and IgG3 in mice. For IgG, the Fc region comprises immunoglobulin domains CH2 and CH3 and the hinge between CH1 and CH2 domains. Although the definition of the boundaries of the Fc region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the Kabat numbering scheme. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fc, including any allotypic variant, or a variant Fc (e.g., a non-naturally-occurring Fc). The methods of the present disclosure are also useful for producing a protein comprising a CTLA4 domain. The methods of the present disclosure are also useful for producing a CTLA4 domain fused to an Fc portion. In some aspects, the protein is a fusion protein. In some aspects, the fusion protein comprises an Fc portion. In some aspects, the protein is belatacept. In some aspects, the protein comprises a sequence selected from the group consisting of SEQ ID Nos: 2-9. In some aspects, the protein comprises SEQ ID NO: 2. In some aspects, the protein comprises SEQ ID NO: 3. In some aspects, the protein comprises SEQ ID NO: 4. In some aspects, the protein comprises SEQ ID NO: 5. In some aspects, the protein comprises SEQ ID NO: 6. In some aspects, the protein comprises SEQ ID NO: 7. In some aspects, the protein comprises SEQ ID NO: 8. In some aspects, the protein comprises SEQ ID NO: 9.

The CTLA4-Ig fusion protein produced by the present methods can comprise one or more mutations. In some aspects, the CTLA4-Ig fusion protein is (a) a CTLA4-Ig fusion protein having an amino acid sequence of SEQ ID NO: 8 (methionine at amino acid position 27 and glycine at amino acid position 382); (b) a CTLA4-Ig fusion protein having an amino acid sequence of SEQ ID NO: 5 (methionine at amino acid position 27 and lysine at amino acid position 383); (c) a CTLA4-Ig fusion protein having an amino acid sequence of SEQ ID NO: 7 (alanine at amino acid position 26 and glycine at amino acid position 382); (d) a CTLA4-Ig fusion protein having an amino acid sequence of SEQ ID NO: 4 (alanine at amino acid position 26 and lysine at amino acid position 383); (e) a CTLA4-Ig fusion protein having an amino acid sequence of SEQ ID NO: 6 (methionine at amino acid position 25 and glycine at amino acid position 382); or (f) a CTLA4-Ig fusion protein having an amino acid sequence of SEQ ID NO: 3 (methionine at amino acid position 25 and lysine at amino acid position 383). In some aspects, the CTLA4-Ig fusion proteins are (a) about 90% of the CTLA4-Ig polypeptides comprise an amino acid sequence of SEQ ID NO: 2 beginning with the methionine at residue 27; (b) about 10% of the CTLA4-Ig polypeptides comprise the amino acid sequence of SEQ ID NO: 2 beginning with the alanine at residue number 26; (c) about 4% of the CTLA4-Ig polypeptides comprise the amino acid sequence of SEQ ID NO: 2 ending with the lysine at residue number 383, (d) about 96% of the CTLA4-Ig polypeptides comprise the amino acid sequence of SEQ ID NO: 2 ending with the glycine at residue number 382; and optionally, (e) about less than 1% of the CTLA4-Ig polypeptides comprise the amino acid sequence of SEQ ID NO: 2 beginning with the methionine at residue number 25.

The proteins of the present disclosure have glycosylation sites. Glycosylation is a process involving the addition of complex oligosaccharide structures to a protein at specific sites within the polypeptide chain. Glycosylation of proteins and the subsequent processing of the added carbohydrates can affect protein folding and structure, protein stability, including protein half-life, and functional properties of a protein. Protein glycosylation can be divided into two classes by virtue of the sequence context where the modification occurs, O-linked glycosylation and N-linked glycosylation. O-linked polysaccharides are linked to a hydroxyl group, usually to the hydroxyl group of either a serine or a threonine residue. O-glycans are not added to every serine and threonine residue. O-linked oligosaccharides are usually mono or biantennary, i.e., they comprise one or at most two branches (antennas), and comprise from one to four different kinds of sugar residues, which are added one by one. N-linked polysaccharides are attached to the amide nitrogen of an asparagine. Only asparagines that are part of one of two tripeptide sequences, either asparagine-X-serine or asparagine-X-threonine (where X is any amino acid except proline), are targets for glycosylation. N-linked oligosaccharides can have from one to four branches referred to as mono-, bi-, tri-tetraantennary. In some aspects, the one or more N-linked glycans are located at one or more asparagine residues selected from the group consisting of Asn76, Asn108, and/or Asn207 of belatacept.

In some aspects, the methods comprise culturing a protein under any one of conditions disclosed herein, e.g., a condition of an initial temperature set point between about 35° C. and about 37° C., e.g., about 36° C., a second temperature set point between 32° C. and about 34° C., e.g., about 33° C., and a third temperature set point between about 30° C. and about 32° C., e.g., about 31° C., wherein the protein has N-linked glycans, e.g., G2F, at residue Asn108.

The methods of the present disclosure are also useful for characterizing, analyzing, or controlling the sialic acid content of proteins. In some aspects, the one or more N-linked glycans are sialic acid and have a molar ratio between about 5 to about 9, from about 5.5 to about 8.5, from about 5.8 to about 6.7, from about 5.2 to about 7.5, from about 6 to about 8, from about 6.2 to about 7.4, or from about 5 to about 6. In some aspects, the one or more N-linked glycans are sialic acid and have a molar ratio between about 3 to about 9, from about 3.5 to about 8.5, from about 4.5 to about 7.5, from about 5.5 to about 7.5, from about 5.5 to about 7.4, from about 6.0 to about 7.4, or from about 6.2 to about 7.4. In some aspects, the one or more N-linked glycans are sialic acid and have a molar ratio of NANA between about 4 to about 7. In some aspects, the one or more N-linked glycans are sialic acid and have a molar ratio of NANA between about 5 to about 8. In some aspects, the one or more N-linked glycans are sialic acid and have a molar ratio of NANA between about 6.2 to about 7.4.

The methods of the present disclosure are also useful for analyzing O-linked glycans. In some aspects, the glycosylation profile includes one or more O-linked glycans. In some aspects, the O-linked glycans are located at residues Ser129, Ser130, Ser136, and/or Ser139.

The methods of the present disclosure are also useful for analyzing bi-antennary glycans of a CTLA4-Fc fusion protein, comprising measuring one or more N-linked glycans attached to one or more asparagine residues in the CTLA4 protein, wherein one of the bi-antennary glycans is G2F. In some aspects, one or more N-linked glycans attached to one or more asparagine residues in the CTLA4 protein are measured, wherein one of the bi-antennary glycans is G0F. In some aspects, the bi-antennary glycans are selected from a group consisting of G0F, G1F, G2F, S1G1F, S1G2F, and/or S2G2F. Liquid chromatography can be used to analyze the glycans of the present disclosure. A particular glycoprotein can display heterogeneity of carbohydrates. Heterogeneity can be seen at several levels: glycosylation sites can vary from completely occupied to unoccupied, and any specific site can be populated with many different oligosaccharide structures, wherein each structure can be modified by sialic acid molecules, such as NANA or NGNA.

The carbohydrate content of the protein of the present disclosure can be analyzed by methods known in the art, including methods described in the Examples herein. Several methods are known in the art for glycosylation analysis and are useful in the context of the present disclosure. These methods provide information regarding the identity and the composition of the oligosaccharide attached to the produced peptide. Methods for carbohydrate analysis useful in connection with the present disclosure include, but are not limited to, lectin chromatography; high performance anion-exchange chromatography combined with pulsed amperometric detection (HPAEC-PAD), which uses high pH anion exchange chromatography to separate oligosaccharides based on charge; NMR; Mass spectrometry; HPLC; porous graphitized carbon (GPC) chromatography.

Methods for releasing oligosaccharides include 1) enzymatic methods, which are commonly performed using peptide-N-glycosidase F/endo-α-galactosidase; 2) β-elimination methods, using a harsh alkaline environment to release mainly O-linked structures; and 3) chemical methods using anhydrous hydrazine to release both N- and O-linked oligosaccharides. Methods for analysis can comprise one or more of the following steps: 1. dialyzing the sample against deionized water to remove all buffer salts, followed by lyophilization; 2. releasing intact oligosaccharide chains with anhydrous hydrazine; 3. treating the intact oligosaccharide chains with anhydrous methanolic HCl to liberate individual monosaccharides as O-methyl derivatives; 4. N-acetylating any primary amino groups; 5. derivatizing to yield per-O-trimethylsilyl methyl glycosides; 6. Separating the derivatives by capillary gas-liquid chromatography (GLC) on a CP-SIL8 column; 7. identifying individual glycoside derivatives by retention time from the GLC and mass spectroscopy, compared to known standards; and 8. quantifying individual derivatives by FID with an internal standard (13-O-methyl-D-glucose). In some aspects, the bi-antennary glycans are measured via Ultra Performance Liquid Chromatography with fluorescence detection (UPLC-FLR). In some aspects, the Fc domain of the CTLA4-Fc fusion protein is cleaved prior to the measuring. In some aspects, the Fc domain of the CTLA4-Fc fusion protein is not cleaved prior to the measuring. In some aspects, the protein is run through a viral inactivation process. In some aspects, the viral inactivation process is run with 0.5% Triton X-100.

### IV. Pharmaceutical Compositions

The proteins produced by the methods of the present disclosure can be further formulated to be suitable for human administration, e.g., pharmaceutical composition. A composition that is acceptable for pharmaceutical administration, such a composition can include substances that are impurities at a level not exceeding an acceptable level for pharmaceutical administration (such level including an absence of such impurities), and can include pharmaceutically acceptable excipients, vehicles, carriers and other inactive ingredients, for example, to formulate such composition for ease of administration, in addition to any active agent(s). For example, a pharmaceutically acceptable CTLA4-Ig composition can include MCP-1 or DNA, so long as those substances are at a level acceptable for administration to humans.

The disclosure also provides any of the described CTLA4-Ig molecules as a lyophilized mixture. Formulations comprising CTLA4-Ig to be lyophilized can further comprise three basic components: (1) an additional active ingredient(s) including other proteins or small molecules (such as immunosuppressants), (2) an excipient(s) and (3) a solvent(s). Excipients include pharmaceutically acceptable reagents to provide good lyophilized cake properties (bulking agents) as well as to provide lyoprotection and/or cryoprotection of proteins (“stabilizer”), maintenance of pH (buffering agents), and proper conformation of the protein during storage so that substantial retention of biological activity (including active ingredient stability, such as protein stability) is maintained. With respect to excipients, an example of a formulation can include one or more of a buffering agent(s), a bulking agent(s), a protein stabilizer(s) and an antimicrobial(s). Sugars or polyols can be used as nonspecific protein stabilizers in solution and during freeze-thawing and freeze-drying. Polymers can be used to stabilize proteins in solution and during freeze-thawing and freeze-drying. One popular polymer is serum albumin, which has been used both as a cryoprotectant and lyoprotectant. In one aspect, the disclosure provides formulations that are albumin free. Various salts can be used as bulking agents. Illustrative salt bulking agents include, for example, NaCl, MgCl2 and CaCl2).

Certain amino acids can be used as cryoprotectants and/or lyoprotectants and/or bulking agents. Amino acids that can be used include, but are not limited to, glycine, proline, 4-hydroxyproline, L-serine, sodium glutamate, alanine, arginine and lysine hydrochloride. Many buffering agents covering a wide pH range are available for selection in formulations. Buffering agents include, for example, acetate, citrate, glycine, histidine, phosphate (sodium or potassium), diethanolamine and Tris. Buffering agents encompasses those agents, which maintain the solution pH in an acceptable range prior to lyophilization. In one aspect, the disclosure provides a lyophilized CTLA4-Ig mixture comprising at least 90%, 95%, 99%, or 99.5% CTLA4-Ig dimer, including any sequence according to any one of SEQ ID Nos: 1-9. In one aspect, the disclosure provides a lyophilized CTLA4-Ig mixture comprising at least 90%, 95%, 99%, or 99.5% CTLA4-Ig dimer and not more than 5%, 4%, 3%, 2%, or 1% CTLA4-Ig tetramer. In another aspect, the disclosure provides a lyophilized CTLA4-Ig mixture comprising at least 90%, 95%, 99%, or 99.5% CTLA4-Ig dimer, and not more than 5%, 4%, 3%, 2%, or 1% CTLA4-Ig tetramer, and not more than 2%, 1.5%, 1.0%, 0.8%, 0.5%, or 0.3% CTLA4-Ig monomer. In a further aspect, the disclosure provides a lyophilized CTLA4-Ig mixture comprising at least 8.0 moles of sialic acid per mole of CTLA4-Ig dimer or to CTLA4-Ig molecule. In another aspect, the disclosure provides a lyophilized CTLA4-Ig mixture comprising: from about 15 to about 35 moles of GlcNac per mole of CTLAIg molecules or dimer; from about 1 to about 5 moles of GalNac per mole of CTLA4-Ig dimer or to CTLA4-Ig molecule; from about 5 moles to about 20 moles of galactose per mole of CTLA4-Ig dimer or to CTLA4-Ig molecule; from about 2 to about 10 moles of fucose per mole of CTLA4-Ig dimer or to CTLA4-Ig molecule; and/or from about 5-15 moles of mannose per mole of CTLA4-Ig dimer or to CTLA4-Ig molecule.

In some aspects, the pharmaceutical composition comprising CTLA4-Ig molecule, e.g., belatacept, can be supplied as a sterile, white or off-white lyophilized powder for intravenous administration. The lyophile can be reconstituted with a suitable fluid to obtain is a clear to slightly opalescent, colorless to pale yellow solution, with a pH in the range of 7.2 to 7.8. Suitable fluids for constitution of the lyophile include SWFI, 0.9% NS, or D5W. Each single-use vial of the CTLA4-Ig molecule, belatacept, also can contain: monobasic sodium phosphate (34.5 mg), sodium chloride (5.8 mg), and sucrose (500 mg).

### V. Methods of Treatment

The compositions prepared by the methods of the present disclosure are useful to treat a variety of diseases. The disclosure provides for a method for inhibiting T cell proliferation (or activation), the method comprising contacting a T cell with an effective amount of a CTLA4-Ig composition of the disclosure. The disclosure provides for a method for inhibiting an immune response in a subject, the method comprising administering to a subject in need thereof an effective amount of a CTLA4-Ig composition of the disclosure. The disclosure provides for a method for inducing immune tolerance to an antigen in a subject, the method comprising administering to a subject in need thereof an effective amount of a CTLA4-Ig composition of the disclosure. The disclosure provides for a method for treating inflammation in a subject, the method comprising administering to a subject in need thereof an effective amount of a CTLA4-Ig composition of the disclosure. The disclosure provides for a method for treating rheumatoid arthritis comprising administering to a subject in need thereof an effective amount of a CTLA4-Ig composition of the disclosure.

The disclosure provides for a method for treating psoriasis in a subject, the method comprising administering to a subject in need thereof an effective amount of a CTLA4-Ig composition of the disclosure. The disclosure provides for a method for treating or preventing an allergy in a subject, the method comprising administering to a subject in need thereof an effective amount of a CTLA4-Ig composition of the disclosure. The disclosure provides for a method for treating or preventing graft vs host disease in a subject, the method comprising administering to a subject in need thereof an effective amount of a CTLA4-Ig composition of the disclosure. The disclosure provides for a method for treating or preventing rejection of a transplanted organ in a subject, the method comprising administering to a subject in need thereof an effective amount of a CTLA4-Ig composition of the disclosure.

The disclosure provides for a method for treating Crohn's Disease in a subject, the method comprising administering to a subject in need thereof an effective amount of a CTLA4-Ig composition of the disclosure. The disclosure provides a method for treating type I diabetes in a subject, the method comprising administering to a subject in need thereof an effective amount of a CTLA4-Ig composition of the disclosure.

The disclosure provides a method for treating oophoritis in a subject, the method comprising administering to a subject in need thereof an effective amount of a CTLA4-Ig composition of the disclosure. The disclosure provides a method for treating glomerulonephritis in a subject, the method comprising administering to a subject in need thereof an effective amount of a CTLA4-Ig composition of the disclosure. The disclosure provides a method for treating allergic encephalomyelitis in a subject, the method comprising administering to a subject in need thereof an effective amount of a CTLA4-Ig composition of the disclosure.

The disclosure provides a method for treating myasthenia gravis in a subject, the method comprising administering to a subject in need thereof an effective amount of a CTLA4-Ig composition of the disclosure. Thus, in certain aspects of the disclosure, the disclosure provides CTLA4-Ig molecules produced by a cell line in a production method described herein in order to treat T-cell related diseases or disorders, that include but are not limited to, generally any T-cell dependent lymphoproliferative disease or disorder and any T-cell dependent autoimmune disease or disorder, and more specifically: T cell lymphoma, T cell acute lymphoblastic leukemia, testicular angiocentric T cell lymphoma, benign lymphocytic angiitis, graft versus host disease (GVHD), immune disorders associated with graft transplantation rejection, psoriasis, inflammation, allergy, oophoritis, glomerulonephritis, encephalomyelitis, Hashimoto's thyroiditis, Graves' disease, Addison's disease, primary myxedema, pernicious anemia, autoimmune atrophic gastritis, rheumatoid arthritis, insulin dependent diabetes mellitis, good pasture's syndrome, myasthenia gravis, pemphigus, sympathetic ophthalmia, autoimmune uveitis, autoimmune hemolytic anemia, idiopathic thrombocytopenia, primary biliary cirrhosis, chronic action hepatitis, scleroderma, polymyositis, and mixed connective tissue disease.

The disclosure provides for a method for inhibiting T cell proliferation (or activation), the method comprising contacting a T cell with an effective amount of a CTLA4-Ig composition of the disclosure in combination with or without another agent, such as methotrexate. The disclosure provides a method for inhibiting an immune response in a subject, the method comprising administering to a subject in need thereof an effective amount of a CTLA4-Ig composition of the disclosure either alone or in combination with methotrexate. The disclosure provides a method for inducing immune tolerance to an antigen in a subject, the method comprising administering to a subject in need thereof an effective amount of a CTLA4-Ig composition of the disclosure in combination with methotrexate.

In some aspects, the CTLA4-Ig molecule, e.g., belatacept, is indicated for prophylaxis of organ rejection in adult patients receiving a kidney transplant. In some aspects, the CTLA4-Ig molecule, e.g. belatacept, is to be used in combination with basiliximab induction, mycophenolate mofetil, and corticosteroids. In some aspects, the CTLA4-Ig molecule, e.g. belatacept, is used only in patients who are EBV seropositive.

Various aspects of the disclosure are described in further detail in the following subsections. The present disclosure is further illustrated by the following examples which should not be construed as further limiting.

### EXAMPLES

### Example 1

### Processes A and B Reference Method

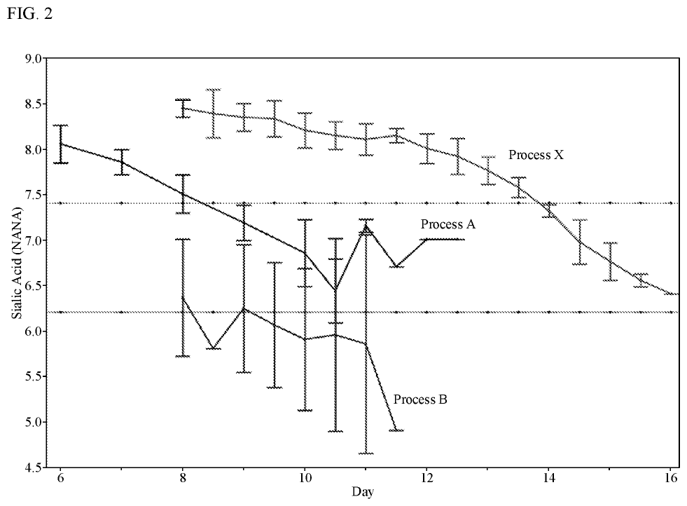

Belatacept is a genetically engineered fusion protein, which consists of the functional binding domain of human Cytotoxic T-Lymphocyte Antigen-4 (CTLA-4) and the Fc domain of human monoclonal immunoglobulin of the IgG1 class. Two amino acid modifications, leucine to glutamic acid at Position 104 and alanine to tyrosine at Position 29 were made in the B7 binding region of the CTLA-4 domain to generate belatacept. Belatacept is comprised of 2 homologous glycosylated polypeptide chains of approximately 46 kDa each of which are covalently linked through a single inter-chain disulfide bond. Belatacept was produced in large-scale cell culture using a Chinese hamster ovary (CHO) cell line and was initiated with the thaw of a frozen vial from an MWCB. The culture was propagated in a series of shake flask cultures. These cultures were then transferred to cell bag bioreactors to generate sufficient cell numbers to inoculate a series of seed bioreactors followed by the production bioreactor. The production bioreactor was harvested based primarily on a target sialic acid (SA) to belatacept protein molar ratio. Finally, the cell culture harvest was clarified in preparation for downstream processing. Representative growth conditions and processes can be seen in FIGS. 1A, 1, and 1C. FIG. 1A shows the reactor conditions for the process for Processes A and B under header “Process A” and “Process B”. FIG. 1B shows the feed timing for the upstream bioreactor growth for Process A and Process B, and FIG. 1C shows the feeding strategy parameters for Process A and Process B. The results of the sialic acid molar ratio analysis for Processes A and B can be seen in FIG. 2. Process A and Process B resulted in higher protein titers, but resulted in significantly lower sialic acid (NANA) molar ratios during production, as seen in FIG. 2. Because the sialic acid molar ratio is a critical quality attribute for the production of Belatacept, Process X was subsequently developed.

### Example 2

### Process C Reference Method

After seed culture expansion, Belatacept is produced in Process C a 5000-L production bioreactor using an initial cell density of 0.8×10⁶ cells/mL, a temperature setting of 37° C., and a pH set point of about 7.05. After the culture has reached a culture time of 144 hours, the bioreactor temperature setpoint is lowered from 37° C. to 34° C. The acceptable range of sialic acid to Belatacept molar ratio at harvest is about 5.8 to about 6.7, and Belatacept is produced at a titer of about 0.34 g/L to about 0.82 g/L.

### Example 3

### CTLA4-Ig 5,000 L Bioreactor Production Process—Process X

In order to maintain the glycosylation profile shown in Process C, but improve the protein yield and/or titer, we have generated Process X as shown herein. Table 1 and Table 2 below summarize the upstream process and in-process controls (IPC) defined for production bioreactor steps of belatacept. Critical process parameters (CPPs), process parameters (PPs), critical process attributes (CPAs), and performance attributes (PAs) are categorized for criticality by a strategy utilizing a tiered set of in-process operating ranges with either acceptable ranges, action ranges, action limits, or alert ranges to ensure the consistent monitoring and control of the drug substance manufacturing process.

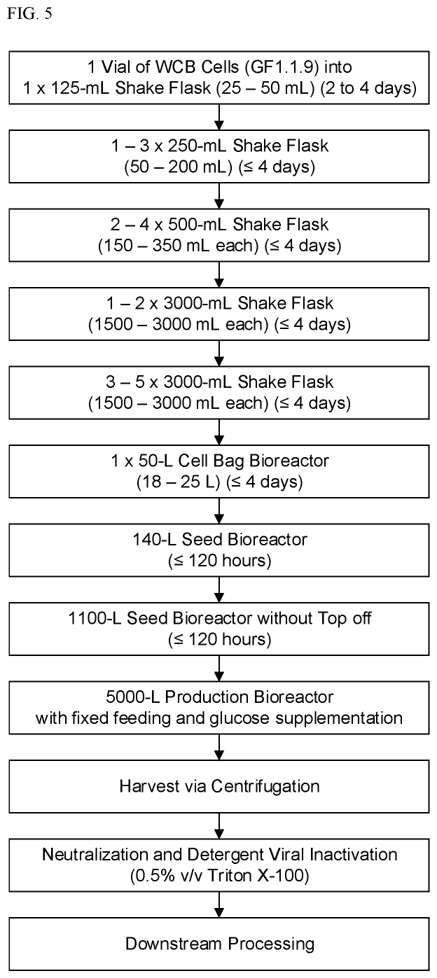

The seed bioreactor steps of the belatacept manufacturing described in Table 1 provide sufficient cell culture biomass to inoculate the production bioreactor. Daily samples were withdrawn from the seed bioreactors to monitor cell growth, percent cell viability and metabolite and nutrient concentration. Robust growth and viability of the cell culture used to inoculate the production bioreactor are important to ensure the consistent production of the belatacept fusion protein. The general process flow for the seed and production bioreactor steps for belatacept manufacturing are shown in FIG. 5.

TABLE 1
Seed Reactor

| Processing Step | Parameter |
|---|---|
| WCB Thaw | Shake Flask |
| Culture Expansion | Shake Flask and Cell Bags |
| 1st Seed Bioreactor | 140-L Stirred Tank |
| 2nd Seed Bioreactor | 1100-L without medium top-off |
| Production Bioreactor | 5000 L Production Bioreactor fed batch |
| | with two temperature shifts. Target |
| | Seeding Density 700,000 viable cels/mL |
| Primary Recovery | Centrifugation, depth filtration, 0.2 μm |
| | membrane filtration, pH adjustment only |

The reactor conditions used during production in the Production Bioreactor stage are described below in Table 2 and FIGS. 1A and 1B. Specifically, the initial temperature was set to 36° C. in order to maintain high cell viability and sialic acid (NANA) content. The Production Bioreactor was adjusted to a pH setpoint of 7.15±0.1 in order to increase growth and sialic acid (NANA) content. During production, the temperature was adjusted down to about 33° C. at approximately 240 hours after induction of the Production Bioreactor, as detailed in FIG. A and Table 2. This temperature adjustment was performed in order to maintain titer over the full production run in the Production Bioreactor. Additionally, the temperature was further reduced to 31° C. at approximately 240 hours after induction, as described in FIG. 1A and Table 2, in order to further maintain titer over the course of the production run. Culture harvest was performed at approximately 240-420 hours, in order to ensure the desired sialic acid (NANA) molar ratio range of 6.2 to 7.4 is reached, as described in FIG. 2. Further reaction parameters are detailed in Table 2. Process X showed production titer greater than 2.0 g/L, that is at least 2 fold higher than the titer by Process C (see Example 2) and Process X produced protein with a much higher sialic acid molar ratio (NANA) as compared to Process A and Process B, as seen in FIG. 2.

TABLE 2
Production Bioreactor Conditions.

| Classification | Process Variable | Criticality | Set point/Target | Action Range |
|---|---|---|---|---|
| Process Input | Initial VCD (×10⁶ | Critical Process | 0.7 | N/A |
| | cells/mL) | Parameter (CPP) | | |
| Process Input | Temperature (° C.) | CPP | 36.0 | 34.5-38.5 |
| Process Input | Temperature Shift 1 | Process Parameter | 140 | N/A |
| | Timing | (PP) | | |
| Process Input | Temperature Shift 1 | CPP | 33 | 6.8-7.5 |
| | (° C.) | | | |
| Process Input | Temperature Shift 2 | PP | 216-264 | N/A |
| | Timing | | | |
| Process Input | Temperature Shift 2 | CPP | 31 | 29.5-33.5 |
| | (° C.) | | | |
| Process Input | First Feed Timing | CPP | 80 | 66-84 |
| | (Hours) | | | |
| Process Input | Post-Innoculation | MP | 2900-3100 | N/A |
| | Volume (L) | | | |
| Process Input | Pressure (psig) | MP | 1-12* | N/A |
| Process Input | Agitation (rpm) | MP | 56-66 | N/A |
| Process Input | Simethicone** | MP | 4.5 ppm Delivered | N/A |
| | Emulsion Addition | | 7 ± 2 Hours, 100 ± 4 | |
| | (ppm based on post- | | Hours, and 168 ± 4 | |
| | inoculation volume) | | Hours | |
| Process Input | Top Air (SLPM) | MP | 25-250 | N/A |
| Process Input | Bottom Air (SLPM) | MP | 26-41 | N/A |
| Process Output | Final Sialic Acid | Critial Performance | 6.7 | 6.2-7.4 |
| | | Attribute (CPA) | | |
| Process Output | Culture Duration | MP | 336 | 240-420 |
| | (Hours) | | | |
| Process Output | Final titler (g/L) | Performance | N/A | ≥2.0 |
| | | Attribute (PA) | | |

*During inoculation and 15 minutes post inoculation, pressure can exceed 12 psig.
**An additional 4 supplemental additions of 4.5 ppm can be added as needed to reduce foaming of the cell culture.

### Example 4

### Glycosylation Analysis of Belatacept—Process X

An immunoglobulin-degrading enzyme from Streptococcus pyogenes (IdeS) is a unique enzyme that digests the hinge region of IgG, at one specific site just below the hinge region, resulting in a CTLA4 fragment and two Fc fragments for belatacept. Complete protein digestion of belatacept drug substance (DS) occurs within 1 hour at 37° C. The digested sample was then purified by elution using a Protein A Spin Column. The resin binds the Fc fragment while allowing the CTLA4 fragment to flow though. The CTLA4 fragment is prepared using Agilent Gly-X® InstantPC N-glycan release and fluorescent labelling kit, and analyzed on Ultra Performance Liquid Chromatography system with fluorescence detection (UPLC-FLR).

UPLC is a chromatographic technique that operates at a higher pressure than high performance liquid chromatography (HPLC), and separation occurs due to the compatibility of the compound and the characteristics of the column being used. In this method, purified belatacept DS CTLA4 region's N-glycans are fluorescently labeled and analyzed by UPLC. The glycans were then separated using a gradient method and an amide column to leverage differences in analyte polarity. The amount of the fluorescently labelled N-glycans in the purified CTLA4 fragment was determined by area %. This method applies to the determination of the amount of fluorescently labeled N-glycans present in the CTLA4 region of belatacept DS samples. Belatacept DS is formulated in 25 mM sodium phosphate, 10 mM sodium chloride, pH 7.5. This method is analyzed on a Waters Acquity UPLC system configured with a Waters Acquity UPLC Glycan BEH Amide, 130 Å 1.7 μm, 2.1 mm×150 mm column, and a Waters Acquity FLR detector. The chromatographic separation parameters for analysis are found in Table 3:

TABLE 3

| Mobile Phase A: | 100% ACN | |
|---|---|---|
| Mobile Phase B: | 50 mM Ammonium Formate, pH 4.4 | |
| Column: | Acquity UPLC Glycan BEH Amide, | |
| | 130 Å, 1.7 μm 2.1 × 150 mm | |
| Sample Temp: | 8° C. ± 4° C. | |
| Column Temp: | 60° C. ± 2° C. | |
| Run Time: | 60 | min |
| Flow Rate: | 0.4 | mL/min |
| Fluorescence Excitation | 285 | mm |
| Fluorescence Emission | 345 | nm |
| UPLC Strong Wash/Seal | 10% ACN/90% H₂O | |
| Wash | | |
| UPLC Weak Wash | 78% ACN/22% Mobile Phase B (50 mM | |
| | Ammonium Formate pH 4.4) | |
| Data Collection Rate | 5 | |
| (pts./sec.) | | |
| PMT Gain | 3 | |
| Injection Volume | 2 | μL |

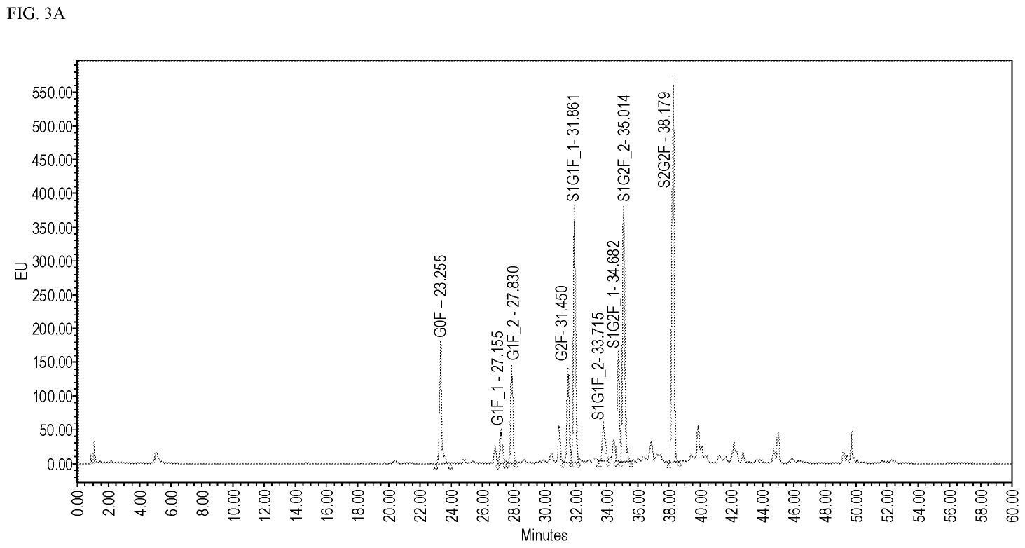

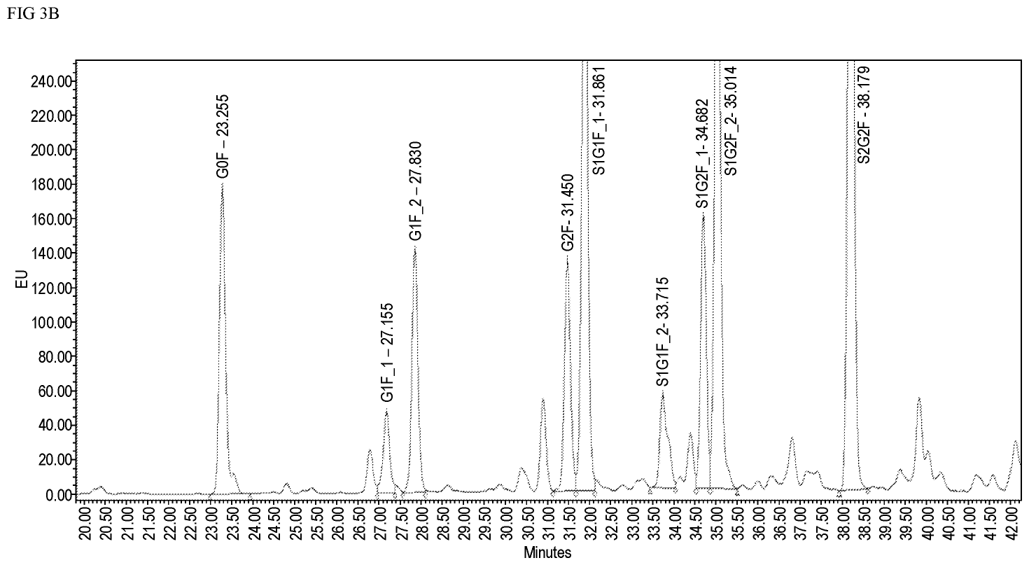

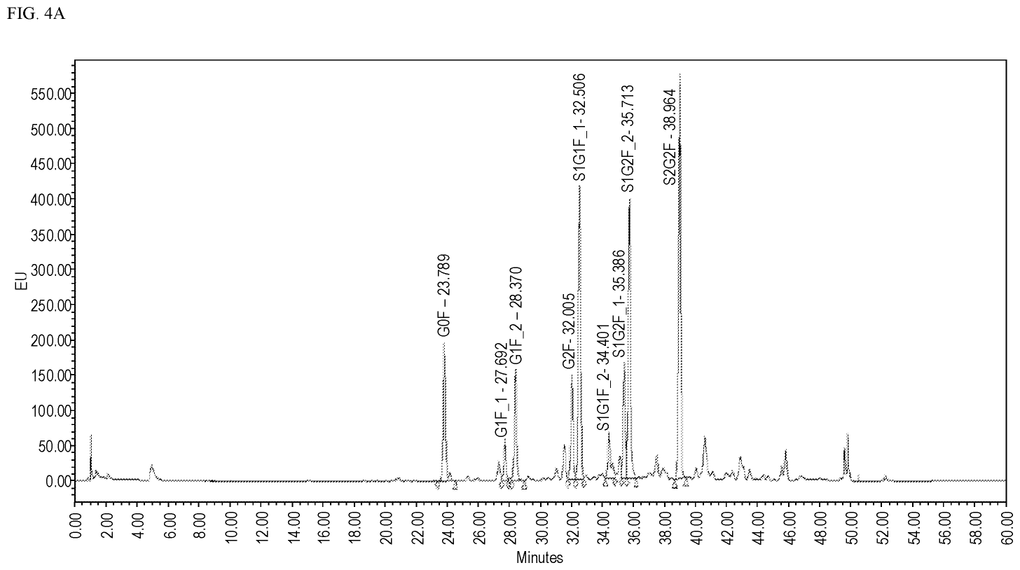

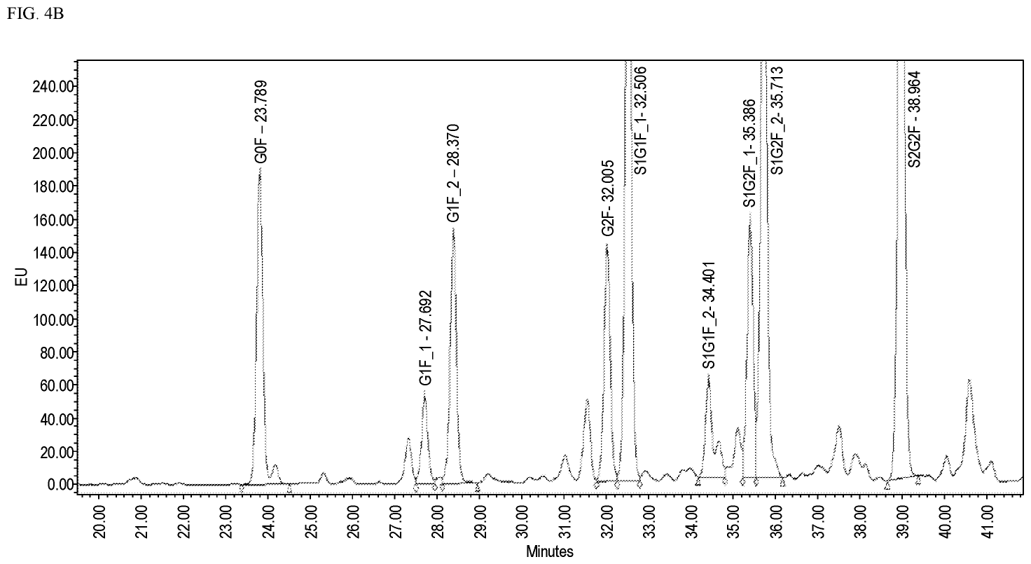

Representative chromatograms of full-scale and zoomed representative chromatograms of the N-glycan profiling of a Belatacept Reference Standard (RS) elution, with specific glycans labeled (G0F, G1F, G2F, S1G1F, and S2G2F) can be seen in FIGS. 3A and 3B, while a full-scale and zoomed representative chromatogram of the N-glycan profiling of a Belatacept RS elution, where glycan S1G1F elutes as two peaks can be seen in FIGS. 4A and 4B. Various glycans are shown in FIGS. 6A and 6B.

Throughout this application, various publications are referenced in parentheses by author name and date, or by patent No. or patent Publication No. The disclosures of these publications are hereby incorporated in their entireties by reference into this application in order to more fully describe the state of the art as known to those skilled therein as of the date of the disclosure described and claimed herein. However, the citation of a reference herein should not be construed as an acknowledgement that such reference is prior art to the present disclosure.

What is claimed is:

1. A method of improving the yield of a protein and/or controlling a glycosylation of the protein during a protein production phase, comprising culturing cells that are capable of expressing the protein in a bioreactor for a protein induction phase under suitable conditions, wherein the suitable conditions comprise a pH set point between about 7.1 and about 7.2.

2. The method of claim 1, wherein the pH set point is about 7.15.

3. The method of claim 1 or 2, wherein the suitable conditions further comprise an initial temperature set point between about 35° C. and about 37° C., a second temperature set point between about 32° C. and about 34° C., and a third temperature set point between about 30° C. to about 32° C.

4. The method of any one of claims 1 to 3, wherein the suitable conditions further comprise an initial viable cell density (VCD) set point between about 0.5×10⁶ cells/mL and about 1×10⁶ cells/mL.

5. The method of any one of claims 1 to 4, wherein the suitable conditions comprise:
a. an initial temperature set point of about 36° C., a second temperature set point of about 33° C., and a third temperature set point of about 31° C.;
b. a pH set point of about 7.15; and
c. an initial viable cell density (VCD) set point of about 0.70×10⁶ cells/mL.

6. A method of controlling cell growth rate, cell viability, viable cell density and/or titer of cells for producing a protein comprising culturing the cells in a bioreactor for a protein induction phase under a pH set point of about 7.15.

7. The method of claim 6, wherein the suitable conditions further comprise (i) an initial temperature set point of about 36.0° C. and a second temperature set point lower than about 36° C.; (ii) an initial temperature set point lower than about 36.5° C. and a final temperature set point of about 31° C.; or (iii) an initial temperature set point lower than about 36.5° C., a second temperature set point of about 33° C., and a final temperature set point lower than about 33° C.

8. The method of claim 6, wherein the suitable conditions further comprise culturing the cell in an initial temperature set point of about 36° C., a second temperature set point of about 33° C., and a final temperature set point of about 31° C.

9. The method of any one of claims 6 to 8, wherein the suitable conditions further comprise an initial viable cell density (VCD) set point of about 0.70×10⁶ cells/mL.

10. A method of improving the yield of belatacept and/or controlling the glycosylation of belatacept during a protein production phase, comprising culturing cells that are capable of expression belatacept in a bioreactor under suitable conditions, wherein the suitable conditions comprise:
a. an initial temperature set point of about 36° C., a second temperature set point of about 33° C., and a third temperature set point of about 31° C.;
b. a pH set point of about 7.15; and
c. an initial viable cell density (VCD) set point of about 0.70×10⁶ cells/mL.

11. The method of any one of claims 1 to 10, wherein the suitable conditions further comprise a first feed time at about 80 hours.

12. The method of any one of claims 1 to 11, wherein the third or final temperature set point occurs between about 204 and about 276 hours.

13. The method of claim 12, wherein the third or final temperature set point occurs at about 204 hours, about 216 hours, about 228 hours, about 240 hours, about 252 hours, about 264 hours, or about 276 hours after the initial temperature set point.

14. The method of any one of claims 1 to 13, wherein the third final temperature set point is about 31° C. and occurs after about 240 hours.

15. The method of any one of claims 1 to 14, wherein the second temperature set point occurs between about 72 hours and about 168 hours.

16. The method of claim 15, wherein the second temperature set point occurs at about 72 hours, about 78 hours, about 84 hours, about 90 hours, about 96 hours, about 102 hours, about 108 hours, about 114 hours, about 120 hours, about 126 hours, about 132 hours, about 138 hours, about 144 hours, about 150 hours, about 156 hours, about 162 hours, or about 168 hours.

17. The method of any one of claims 1 to 16, wherein the second temperature set point is about 33° C. after about 140 hours.

18. The method of any one of claims 1 to 17, wherein the conditions improve the protein yield by at least 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, or at least about 400%; as compared to a reference method without the suitable conditions.

19. The method of any one of claims 1 to 18, wherein the suitable condition further comprises manganese in the bioreactor.

20. The method of claim 19, wherein the manganese is present at a concentration from about 1.6 parts per billion (ppb) to about 15 ppb.

21. The method of claim 19, wherein the manganese is present at a concentration from about 3 ppb to about 6 ppb.

22. The method of any one of claims 1 to 21, wherein the method reduces cell growth rate.

23. The method of any one of claims 1 to 22, wherein the method controls a cell viability.

24. The method of claim 23, wherein the cell viability exhibits a mean peak viable cell density (VCD) between about 10.0×10⁶ cells/mL and about 15.0×10⁶ cells/mL.

25. The method of any one of claims 1 to 24, wherein the method controls a titer.

26. The method of claim 25, wherein the titer exhibits a final titer between about 1.50 g/L and about 3.5 g/L.

27. The method of claim 25, wherein the titer exhibits a final titer great than about 2.00 g/L.

28. The method of any one of claims 1 to 27, wherein the method controls a glycosylation profile of the protein.

29. The method of claim 28, wherein the glycosylation profile comprises one or more N-linked glycans.

30. The method of claim 28 or 29, wherein the glycosylation profile is measured during the protein production phase.

31. The method of claim 30, wherein the glycosylation profile is measured about every 1 day.

32. The method of any one of claims 29 to 31, wherein the glycosylation profile is measured when the cell culture is harvested.

33. The method of any one of claims 29 to 32, wherein the N-linked glycans comprise: G0F, G1F, G2F, S1G1F, S1G2F, S2G2F, or any combination thereof.

34. The method of any one of claims 1 to 9 or 11 to 33, wherein the protein comprises a CTLA4 domain.

35. The method of any one of claims 1 to 9 or 11 to 34, wherein the protein is a fusion protein.

36. The method of claim 35, wherein the fusion protein comprises an Fc portion.

37. The method of any one of claims 1 to 9 or 11 to 36, wherein the protein is belatacept.

38. The method of any one of claims 1 to 37, wherein the protein comprises an amino acid sequence at least about 80%, at least about 85%, at least about 90%, at least about 95%, at least about 96%, at least about 97%, at least about 98%, at least about 99%, or about 100% identical to SEQ ID NO: 5.

39. The method of any one of claims 29 to 38, wherein the one or more N-linked glycans are located at one or more residues selected from the group consisting of Asn76, Asn108, and/or Asn207 of belatacept.

40. The method of any one of claims 29 to 39, wherein the one or more N-linked glycans comprises sialic acid and have a molar ratio of NANA of from about 4 to about 10.

41. The method of any one of claims 29 to 40, wherein the one or more N-linked glycans comprises sialic acid and have a molar ratio of NANA of from about 5 to about 9, from about 5.5 to about 8.5, from about 5.8 to about 6.7, from about 5.2 to about 7.5, from about 6 to about 8, from about 6.2 to about 7.4, or from about 5 to about 6.

42. The method of claim 41, wherein the molar ratio of NANA is about 6.8.

43. The method of any one of claims 28 to 42, wherein the glycosylation profile is analyzed via a N-linked carbohydrate profile method.

44. The method of any one of claims 28 to 43, wherein the glycosylation profile includes one or more O-linked glycans.

45. The method of claim 44, wherein the O-linked glycans are located at residues Ser129, Ser130, Ser136, and/or Ser139.

46. The method of any one of claims 1 to 45, which is performed as a fed-batch culture process.

47. The method of any one of claims 1 to 46, wherein glucose and/or galactose are supplemented to a feed media in the bioreactor.

48. The method of claim 47 wherein the feed media is added to the bioreactor periodically.

49. The method of claim 48, wherein the feed media is added to the bioreactor about every 24 hours.

50. The method of any one of claims 1 to 45, which is performed as a perfusion process.

51. The method of any one of claims 1 to 50, wherein the cells are mammalian cells.

52. The method of claim 51, wherein the mammalian cells are Chinese hamster ovary (CHO) cells.

53. The method of claim 52, wherein the mammalian cells are CHO-K1 cells, CHO-DXB11 cells, or CHO-DG44 cells.

54. A method of analyzing glycans of a CTLA4-Fc fusion protein, comprising measuring one or more N-linked glycans attached to one or more asparagine residues in the CTLA4 protein, wherein one of the glycans comprises G0F, G1F, G2F, S1G1F, S1G2F, and/or S2G2F.

55. The method of claim 54, wherein the glycans are measured via Ultra Performance Liquid Chromatography with fluorescence detection (UPLC-FLR).

56. The method of claim 54 or 55, wherein the Fc domain of the CTLA4-Fc fusion protein is cleaved prior to the measuring.

57. The method of claim 54, wherein the Fc domain of the CTLA4-Fc fusion protein is not cleaved prior to the measuring.

58. A protein produced by the method of any one of claims 1 to 57.

59. A protein produced by the method of any one of claims 1 to 9, and 11 to 57, wherein the protein comprises a CTLA4-Fc fusion protein.

60. The protein of claim 59, wherein the protein is belatacept.

61. A cell produced by the method of any one of claims 1 to 57.

62. A cell produced by the method of any one of claims 1 to 50 and 54 to 57, wherein the cell is a mammalian cell.

63. The cell of claim 62, wherein the cell is a Chinese hamster ovary (CHO) cell.

64. The cell of claim 63, wherein the cell is a CHO-K1 cell, CHO-DXB11 cell, or CHO-DG44 cell.

65. A bioreactor for the manufacture of a protein produced in the method of any one of claims 1 to 57.

66. A bioreactor comprising the cell of any one of claims 61 to 64 and a cell culture medium, wherein the bioreactor is maintained at a pH of about 7.15.

67. The bioreactor of claim 66, wherein the bioreactor is maintained at:
a. an initial temperature set point of about 36° C., a second temperature set point of about 33° C., and a third temperature set point of about 31° C.;
b. a pH set point of about 7.15; and
c. an initial viable cell density (VCD) set point of about 0.70×10⁶ cells/mL.

68. The bioreactor of claim 66 or 67, wherein the cell culture medium further comprises manganese.


SEQUENCE LISTING (9 sequences)

SEQ ID NO: 1 - protein, 124 aa, Artificial Sequence [sequence omitted]

SEQ ID NO: 2 - protein, 383 aa, Artificial Sequence
  CTLA4A29YL104E-Ig amino acid sequence
MGVLLTQRTLLSLVLALLFPSMASMAMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 359 aa, Artificial Sequence
  amino acids 25-383 of SEQ ID NO 2
MAMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 358 aa, Artificial Sequence
  amino acids 26-383 of SEQ ID NO 2
AMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 357 aa, Artificial Sequence
  amino acids 27-383 of SEQ ID NO 2
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 358 aa, Artificial Sequence
  amino acids 25-382 of SEQ ID NO 2
MAMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 357 aa, Artificial Sequence
  amino acids 26-382 of SEQ ID NO 2
AMHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 356 aa, Artificial Sequence
  amino acids 27-382 of SEQ ID NO 2
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 124 aa, Artificial Sequence
  CTLA4 Extracellular Domain with A29Y and L104E Mutations
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD